(19) United States
(12) Patent Application Publication
(10) Pub. No.: US 2026/0033894 A1
(43) Pub. Date: Feb. 5, 2026

(54) ROBOTIC SURGERY SYSTEM WITH SURFACE EVALUATION IN REVISION PROCEDURES

(71) Applicant: MAKO Surgical Corp., Weston, FL (US)

(72) Inventors: Yoribaldis Olivo, Plantation, FL (US); Kunal Bhargava, Delhi (IN); Harsh Jain, Dhrangadhra (IN); Vaibhav Kumar, Ludhiana (IN); Varun Chandra, Fort Lauderdale, FL (US); Matthew Thompson, Durham, NC (US)

(21) Appl. No.: 19/273,578

(22) Filed: Jul. 18, 2025

Related U.S. Application Data
(60) Provisional application No. 63/677,125, filed on Jul. 30, 2024.

(51) Int. Cl. A61B 34/10; A61B 17/16; A61B 34/20; A61B 34/30; A61F 2/32

(52) U.S. Cl. CPC A61B 34/10; A61B 17/1615; A61B 34/20; A61B 34/30; A61F 2/32; A61B 2034/102; A61B 2034/105; A61B 2034/2055; A61B 2034/2065

(57) ABSTRACT

A method includes obtaining a surgical plan including a removal of a first physical implant from a bone and a replacement of the first physical implant with a second physical implant. The method also includes receiving a first virtual model including a first virtual implant model relative to a first virtual bone surface of a bone. The first virtual bone surface represents a first bone surface to which the first physical implant is coupled. The method also includes identifying a second bone surface resulting from removal of the first physical implant from the bone by tracking a position of a surgical tool relative to the bone. The method also includes detecting a discrepancy between the first bone surface and the second bone surface and displaying, on a graphical user interface, a visualization of the discrepancy.

### CROSS-REFERENCE TO RELATED APPLICATION

This application claims the benefit of and priority to U.S. Provisional Application No. 63/677,125, filed Jul. 30, 2024, the entire disclosure of which is incorporated by reference herein.

### BACKGROUND

The present disclosure relates generally to surgical systems for orthopedic surgeries, and more particularly to surgical systems for total and partial hip arthroplasty procedures. Hip arthroplasty, colloquially referred to as hip replacement, is widely used to treat hip osteoarthritis and other damage to a patient's hip joint by replacing portions of the hip anatomy with prosthetic components.

One possible tool for use in total hip arthroplasty procedure is a robotically-assisted surgical system. A robotically-assisted surgical system typically includes a robotic device that is used to prepare a patient's anatomy, a tracking system configured to monitor the location of the robotic device relative to the patient's anatomy, and a computing system configured to monitor and control the robotic device. Robotically-assisted surgical systems, in various forms, autonomously carry out surgical tasks, provide force feedback to a user manipulating a surgical device to complete surgical tasks, augment surgeon dexterity and precision, and/or provide other navigational cues to facilitate safe and accurate surgical operations.

A surgical plan is typically established prior to performing a surgical procedure with a robotically-assisted surgical system. Based on the surgical plan, the surgical system guides, controls, or limits movements of the surgical tool during portions of the surgical procedure. Guidance and/or control of the surgical tool serves to protect the patient and to assist the surgeon during implementation of the surgical plan.

### SUMMARY

One implementation of the present disclosure is a method. The method includes obtaining a surgical plan including a removal of a first physical implant from a bone and a replacement of the first physical implant with a second physical implant; receiving a first virtual model including a first virtual implant model relative to a first virtual bone surface of a bone, where the first virtual bone surface represents a first bone surface to which the first physical implant is coupled; identifying a second bone surface resulting from removal of the first physical implant from the bone by tracking a position of a surgical tool relative to the bone; detecting a discrepancy between the first bone surface and the second bone surface; and displaying, on a graphical user interface, a visualization of the discrepancy.

Another implementation of the present disclosure is a system. The system includes a robotic device and a circuitry. The circuitry is configured to obtain a surgical plan including a removal of a first physical implant from a bone and a replacement of the first physical implant with a second physical implant; receive a first virtual model including a first virtual implant model relative to a first virtual bone surface of a bone, where the first virtual bone surface represents a first bone surface to which the first physical implant is coupled; identify a second bone surface resulting from removal of the first physical implant from the bone by tracking a position of a surgical tool relative to the bone; detect a discrepancy between the first bone surface and the second bone surface; and display, on a graphical user interface, a visualization of the discrepancy.

Another implementation of the present disclosure relates to one or more non-transitory computer-readable media storing instructions that, when executed by a processor, cause the processor to perform operations. The operations include obtaining a surgical plan including a removal of a first physical implant from a bone and a replacement of the first physical implant with a second physical implant; receiving a first virtual model including a first virtual implant model relative to a first virtual bone surface of a bone, where the first virtual bone surface represents a first bone surface to which the first physical implant is coupled; identifying a second bone surface resulting from removal of the first physical implant from the bone by tracking a position of a surgical tool relative to the bone; detecting a discrepancy between the first bone surface and the second bone surface; and displaying, on a graphical user interface, a visualization of the discrepancy.

### BRIEF DESCRIPTION OF THE DRAWINGS

FIG. 1A is a perspective view of a femur and a pelvis.

FIG. 1B is a perspective view of a hip joint formed by the femur and pelvis of FIG. 1A.

FIG. 1C is an exploded perspective view of a femoral component and an acetabular component for a total hip replacement procedure.

FIG. 1D is a perspective view illustrating placement of the femoral component and acetabular component of FIG. 1C in relation to the femur and pelvis of FIG. 1A, respectively.

FIG. 2 is an illustration of a surgical system, according to an exemplary embodiment.

FIG. 3 is a flowchart of a process for facilitating an arthroplasty procedure, according to an exemplary embodiment.

FIG. 4 is a first illustration of a graphical user interface that can be used with the process of FIG. 3, according to an exemplary embodiment.

FIG. 5 is a second illustration of a graphical user interface that can be used with the process of FIG. 3, according to an exemplary embodiment.

FIG. 6 is a third illustration of a graphical user interface that can be used with the process of FIG. 3, according to an exemplary embodiment.

FIG. 7 is a fourth illustration of a graphical user interface that can be used with the process of FIG. 3, according to an exemplary embodiment.

FIG. 8 is a flowchart of showing a detailed view of steps of the process of FIG. 3, according to an exemplary embodiment.

FIG. 9 is a first visualization of registration regions on a pelvis for use with the process of FIG. 3, according to an exemplary embodiment.

FIG. 10 is a second visualization of registration regions on a pelvis for use with the process of FIG. 3, according to an exemplary embodiment.

FIG. 11 is a flowchart of a process for mapping a bone surface of a defect cavity during the process of FIG. 3, according to an exemplary embodiment.

FIG. 12 is a detailed view of parts of the surgical system of FIG. 2 used to perform the process of FIG. 11, according to an exemplary embodiment.

FIG. 13 is a flowchart of a process for mapping a bone surface of a defect cavity using a probe during the process of FIG. 11, according to an exemplary embodiment.

FIG. 14 is a first illustration of a graphical user interface that can be used with the process of FIG. 13, according to an exemplary embodiment.

FIG. 15 is a second illustration of a graphical user interface that can be used with the process of FIG. 13, according to an exemplary embodiment.

FIG. 16 is a flowchart of a process for mapping a bone surface of a defect cavity using a single-capture reamer mode during the process of FIG. 11, according to an exemplary embodiment.

FIG. 17 is a depiction of a virtual bone model of a defect cavity as displayed during the process of FIG. 16, according to an exemplary embodiment.

FIG. 18 is a flowchart of a process for mapping a bone surface of a defect cavity using a multi-capture reamer mode during the process of FIG. 11, according to an exemplary embodiment.

FIG. 19 is a depiction of a virtual bone model of a defect cavity as displayed during the process of FIG. 18, according to an exemplary embodiment.

FIG. 20 is a first illustration of a graphical user interface that can be used with the process of FIG. 18, according to an exemplary embodiment.

FIG. 21 is a second illustration of a graphical user interface that can be used with the process of FIG. 18, according to an exemplary embodiment.

FIG. 22 is a third illustration of a graphical user interface that can be used with the process of FIG. 18, according to an exemplary embodiment.

FIG. 23 is a fourth illustration of a graphical user interface that can be used with the process of FIG. 18, according to an exemplary embodiment.

FIG. 24 is a fifth illustration of a graphical user interface that can be used with the process of FIG. 18, according to an exemplary embodiment.

FIG. 25 is a fifth illustration of a graphical user interface that can be used with the process of FIG. 3, according to an exemplary embodiment.

FIG. 26 is a sixth illustration of a graphical user interface that can be used with the process of FIG. 3, according to an exemplary embodiment.

FIG. 27 is a depiction of an implant augment and a probe as used in the process of FIG. 3, according to an exemplary embodiment.

FIG. 28 is a depiction of fixation of the implant augment of FIG. 13 to a bone as in the process of FIG. 3, according to an exemplary embodiment.

FIG. 29 is a depiction of a cup impaction step of the process of FIG. 3, according to an exemplary embodiment.

FIG. 30 is a depiction of a cement curing step of the process of FIG. 3, according to an exemplary embodiment.

### DETAILED DESCRIPTION

Presently preferred embodiments of the invention are illustrated in the drawings. An effort has been made to use the same or like reference numbers throughout the drawings to refer to the same or like parts. Although this specification refers primarily to a robotic arm for orthopedic hip replacement, it should be understood that the subject matter described herein is applicable to other types of robotic systems, including those used for surgical and non-surgical applications, as well as to other joints of the body, such as, for example, a knee or shoulder joint.

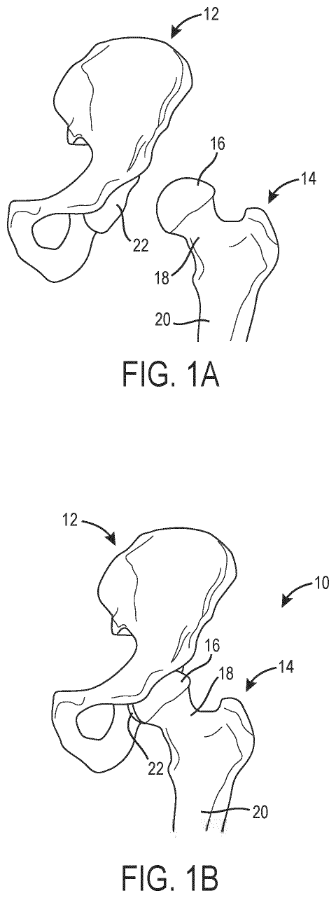

The hip joint is the joint between the femur and the pelvis and primarily functions to support the weight of the body in static (for example, standing) and dynamic (for example, walking) postures. FIG. 1A illustrates the bones of a hip joint 10, which include a pelvis 12 (shown in part) and a proximal end of a femur 14. The proximal end of the femur 14 includes a femoral head 16 disposed on a femoral neck 18. The femoral neck 18 connects the femoral head 16 to a femoral shaft 20. As shown in FIG. 1B, the femoral head 16 fits into a concave socket in the pelvis 12 called the acetabulum 22, thereby forming the hip joint 10. The acetabulum 22 and femoral head 16 are both covered by articular cartilage that absorbs shock and promotes articulation of the joint 10.

Over time, the hip joint 10 may degenerate (for example, due to osteoarthritis) resulting in pain and diminished functionality. As a result, a hip replacement procedure, such as total hip arthroplasty or hip resurfacing, may be necessary. During hip replacement, a surgeon replaces portions of a patient's hip joint 10 with artificial components. In total hip arthroplasty, the surgeon removes the femoral head 16 and neck 18 and replaces the natural bone with a prosthetic femoral component 26 comprising a head 26a, a neck 26b, and a stem 26c (shown in FIG. 1C). As shown in FIG. 1D, the stem 26c of the femoral component 26 is anchored in a cavity the surgeon creates in the intramedullary canal of the femur 14. Alternatively, if disease is confined to the surface of the femoral head 16, the surgeon may opt for a less invasive approach in which the femoral head is resurfaced (e.g., using a cylindrical reamer) and then mated with a prosthetic femoral head cup (not shown).

Similarly, if the natural acetabulum 22 of the pelvis 12 is worn or diseased, the surgeon resurfaces the acetabulum 22 using a reamer and replaces the natural surface with a prosthetic acetabular component 28 comprising a hemispherical shaped cup 28a (shown in FIG. 1C) that may include a liner 28b. To install the acetabular component 28, the surgeon connects the cup 28a to a distal end of an impactor tool and implants the cup 28a into the reamed acetabulum 22 by repeatedly striking a proximal end of the impactor tool with a mallet. If the acetabular component 28 includes a liner 28b, the surgeon snaps the liner 28b into the cup 28a after implanting the cup 28a. Depending on the position in which the surgeon places the patient for surgery, the surgeon may use a straight or offset reamer to ream the acetabulum 22 and a straight or offset impactor to implant the acetabular cup 28a. For example, a surgeon that uses a postero-lateral approach may prefer straight reaming and impaction whereas a surgeon that uses an antero-lateral approach may prefer offset reaming and impaction.

In some cases, an implant augment is used to support or otherwise facilitate reconstruction of the acetabulum 22 to facilitate fixation of the cup 28a to the pelvis 12 in a preferred position and orientation. Use of an augment may be preferable in several scenarios. As one example, an implant augment may be advantageous post-traumatic hip reconstructions, in which a traumatic injury (e.g., car crash, etc.) caused damage to the pelvis 12. As another example, an implant augment may be advantageous in cases of hip dysplasia or other cases of acetabular bone loss, i.e., to fill space created by such bone loss. As another example, an implant augment may be advantageous for revision hip arthroplasty procedures, in which a previously-implanted hip prosthesis is removed and replaced with a new implant due to degradation of neighboring bone or other complications.

Current surgical procedures that involve implant augments typically rely on surgeon expertise and experience to manually place an implant augment in a position that looks and feels correct to the surgeon intraoperatively. Such procedures may be difficult and result in extended surgical time. Additionally, currently-available robotically-assisted surgical devices for hip arthroplasty do not provide for placement of implant augments. The systems and methods described herein provide for computer-assisted planning of implant placement and robotically-assisted surgical steps to facilitate bone preparation for implant augments and placement of implant augments during hip arthroplasty procedures, thereby facilitating hip arthroplasty procedures in cases of bone loss, traumatic injury, revision hip replacements, or other relevant scenarios. The systems and methods described herein may thereby improve patient outcomes, reduce surgery times, and reduce the burden on surgeons for augmented hip arthroplasty procedures.

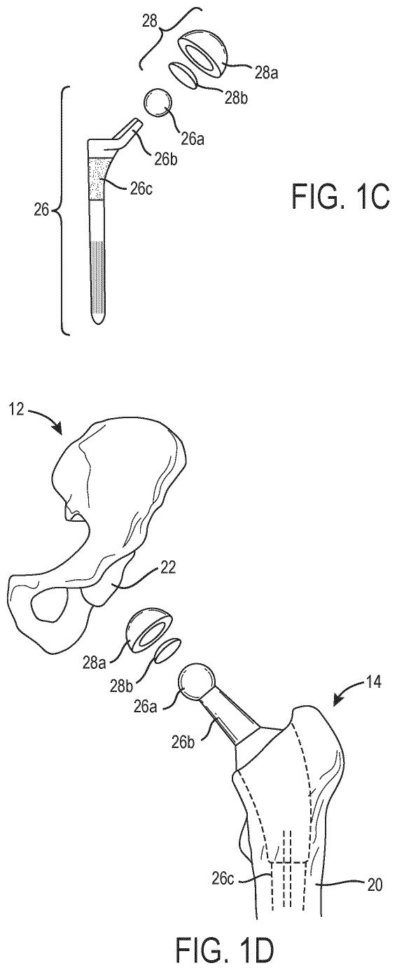

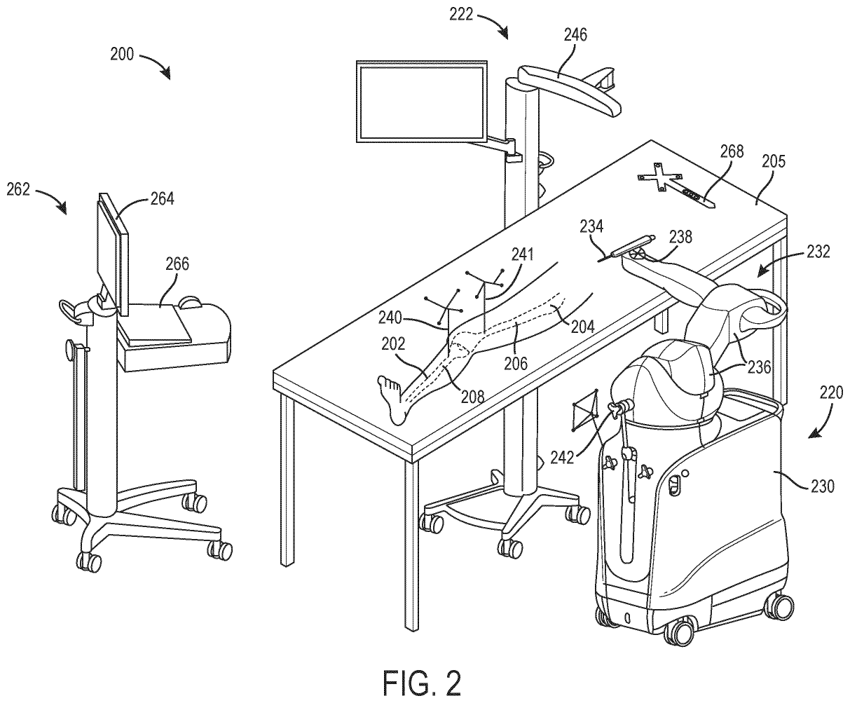

Referring now to FIG. 2, a surgical system 200 for orthopedic surgery is shown, according to an exemplary embodiment. In general, the surgical system 200 is configured to facilitate the planning and execution of a surgical plan, for example to facilitate a joint-related procedure. As shown in FIG. 2, the surgical system 200 is set up to treat a leg 202 of a patient 204 sitting or lying on table 205. In the illustration shown in FIG. 2, the leg 202 includes femur 206 and tibia 208, between which a prosthetic knee implant is to be implanted in a total knee arthroscopy procedure. In other scenarios, for example as described herein with reference to 1A-1D and FIGS. 3-30, the surgical system 200 is set up to treat the hip 10 of a patient, i.e., the femur 14 and the pelvis 12 of the patient (illustrated in FIGS. 1A-1D). Additionally, in still other scenarios, the surgical system 200 is set up to treat a shoulder of a patient, i.e., to facilitate replacement and/or augmentation of components of a shoulder joint (e.g., to facilitate placement of a humeral component, a glenoid component, and a graft or implant augment). Various other anatomical regions and procedures are also possible. To facilitate the procedure, surgical system 200 includes robotic device 220, tracking system 222, and computing system 224.

The robotic device 220 is configured to modify a patient's anatomy (e.g., femur 206 of patient 204) under the control of the computing system 224. One embodiment of the robotic device 220 is a haptic device. “Haptic” refers to a sense of touch, and the field of haptics relates to, among other things, human interactive devices that provide feedback to an operator. Feedback may include tactile sensations such as, for example, vibration. Feedback may also include providing force to a user, such as a positive force or a resistance to movement. One use of haptics is to provide a user of the device with guidance or limits for manipulation of that device. For example, a haptic device may be coupled to a surgical tool, which can be manipulated by a surgeon to perform a surgical procedure. The surgeon's manipulation of the surgical tool can be guided or limited through the use of haptics to provide feedback to the surgeon during manipulation of the surgical tool.

Another embodiment of the robotic device 220 is an autonomous or semi-autonomous robot. “Autonomous” refers to a robotic device's ability to act independently or semi-independently of human control by gathering information about its situation, determining a course of action, and automatically carrying out that course of action. For example, in such an embodiment, the robotic device 220, in communication with the tracking system 222 and the computing system 224, may autonomously complete the series of femoral cuts mentioned above without direct human intervention.

The robotic device 220 includes a base 230, a robotic arm 232, and a surgical tool 234, and is communicably coupled to the computing system 224 and the tracking system 222. The base 230 provides a moveable foundation for the robotic arm 232, allowing the robotic arm 232 and the surgical tool 234 to be repositioned as needed relative to the patient 204 and the table 205. The base 230 may also contain power systems, computing elements, motors, and other electronic or mechanical system necessary for the functions of the robotic arm 232 and the surgical tool 234 described below.

The robotic arm 232 is configured to support the surgical tool 234 and provide a force as instructed by the computing system 224. In some embodiments, the robotic arm 232 allows a user to manipulate the surgical tool and provides force feedback to the user. In such an embodiment, the robotic arm 232 includes joints 236 and mount 238 that include motors, actuators, or other mechanisms configured to allow a user to freely translate and rotate the robotic arm 232 and surgical tool 234 through allowable poses while providing force feedback to constrain or prevent some movements of the robotic arm 232 and surgical tool 234 as instructed by computing system 224. As described in detail below, the robotic arm 232 thereby allows a surgeon to have full control over the surgical tool 234 within a control object while providing force feedback along a boundary of that object (e.g., a vibration, a force preventing or resisting penetration of the boundary). In some embodiments, the robotic arm is configured to move the surgical tool to a new pose automatically without direct user manipulation, as instructed by computing system 224, in order to position the robotic arm as needed and/or complete certain surgical tasks, including, for example, cuts in a femur 206 or an acetabulum.

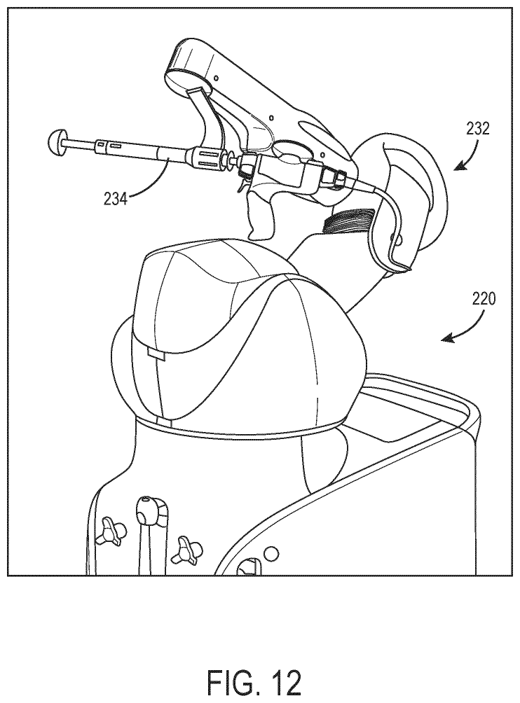

The surgical tool 234 is configured to cut, burr, grind, drill, partially resect, reshape, and/or otherwise modify a bone. In some embodiments, the surgical tool 234 may also be used to trace (i.e., map) a surface of a bone. The surgical tool 234 may be any suitable tool, and may be one of multiple tools interchangeably connectable to robotic device 220. For example, as shown in FIG. 2 the surgical tool 234 is a spherical burr. The surgical tool may also be a sagittal saw, for example with a blade aligned parallel with a tool axis or perpendicular to the tool axis. The surgical tool 234 may also be a holding arm or other support configured to hold an implant component (e.g., cup 28a, implant augment, etc.) in position while the implant component is screwed to a bone, adhered (e.g., cemented) to a bone or other implant component, or otherwise installed in a preferred position. In some embodiments, the surgical tool 234 is an impaction tool configured to provide an impaction force to a cup 28a to facilitate fixation of the cup 28a to a pelvis 12 in a planned location and orientation. The surgical tool 234 may also include a probe 268 and/or a reamer configured to trace an exposed surface of a bone, as described herein. For example, as described below, FIG. 12 illustrates embodiments of the surgical system 200 where the surgical tool 234 is a reamer.

Tracking system 222 is configured to track the patient's anatomy (e.g., femur 206 and tibia 208) and the robotic device 220 (i.e., surgical tool 234 and/or robotic arm 232) to enable control of the surgical tool 234 coupled to the robotic arm 232, to determine a position and orientation of modifications or other results made by the surgical tool 234, and allow a user to visualize the bones (e.g., femur 206, the tibia 208, pelvis 12, humerus, scapula, etc. as applicable in various procedures), the surgical tool 234, and/or the robotic arm 232 on a display of the computing system 224. More particularly, the tracking system 222 determines a position and orientation (i.e., pose) of objects (e.g., surgical tool 234, femur 206) with respect to a coordinate frame of reference and tracks (i.e., continuously determines) the pose of the objects during a surgical procedure. According to various embodiments, the tracking system 222 may be any type of navigation system, including a non-mechanical tracking system (e.g., an optical tracking system), a mechanical tracking system (e.g., tracking based on measuring the relative angles of joints 236 of the robotic arm 232), or any combination of non-mechanical and mechanical tracking systems.

In the embodiment shown in FIG. 2, the tracking system 222 includes an optical tracking system. Accordingly, tracking system 222 includes a first fiducial tree 240 coupled to the tibia 208, a second fiducial tree 241 coupled to the femur 206, a third fiducial tree 242 coupled to the base 230, one or more fiducials coupled to surgical tool 234, and a detection device 246 configured to detect the three-dimensional position of fiducials (i.e., markers on fiducial trees 240-242). Fiducial trees 240, 241 may be coupled to other bones as suitable for various procedures (e.g., pelvis 12 and femur 206 in a hip arthroplasty procedure). Detection device 246 may be an optical detector such as a camera or infrared sensor. The fiducial trees 240-242 include fiducials, which are markers configured to show up clearly to the optical detector and/or be easily detectable by an image processing system using data from the optical detector, for example by being highly reflective of infrared radiation (e.g., emitted by an element of tracking system 222). A stereoscopic arrangement of cameras on detection device 246 allows the position of each fiducial to be determined in 3D-space through a triangulation approach. Each fiducial has a geometric relationship to a corresponding object, such that tracking of the fiducials allows for the tracking of the object (e.g., tracking the second fiducial tree 241 allows the tracking system 222 to track the femur 206), and the tracking system 222 may be configured to carry out a registration process to determine or verify this geometric relationship. Unique arrangements of the fiducials in the fiducial trees 240-242 (i.e., the fiducials in the first fiducial tree 240 are arranged in a different geometry than fiducials in the second fiducial tree 241) allow for distinguishing the fiducial trees, and therefore the objects being tracked, from one another.

Using the tracking system 222 of FIG. 2 or some other approach to surgical navigation and tracking, the surgical system 200 can determine the position of the surgical tool 234 relative to a patient's anatomical feature, for example femur 206, as the surgical tool 234 is used to modify the anatomical feature or otherwise facilitate the surgical procedure. Additionally, using the tracking system 222 of FIG. 2 or some other approach to surgical navigation and tracking, the surgical system 200 can determine the relative poses of the tracked bones.

The computing system 224 is configured to create a surgical plan, control the robotic device 220 in accordance with the surgical plan to make one or more bone modifications and/or facilitate implantation of one or more prosthetic components. Accordingly, the computing system 224 is communicably coupled to the tracking system 222 and the robotic device 220 to facilitate electronic communication between the robotic device 220, the tracking system 222, and the computing system 224. Further, the computing system 224 may be connected to a network to receive information related to a patient's medical history or other patient profile information, medical imaging, surgical plans, surgical procedures, and to perform various functions related to performance of surgical procedures, for example by accessing an electronic health records system. Computing system 224 includes processing circuit 260 and input/output device 262.

The input/output device 262 is configured to receive user input and display output as needed for the functions and processes described herein. As shown in FIG. 2, input/output device 262 includes a display 264 and a keyboard 266. The display 264 is configured to display graphical user interfaces generated by the processing circuit 260 that include, for example, information about surgical plans, medical imaging, settings and other options for surgical system 200, status information relating to the tracking system 222 and the robotic device 220, and tracking visualizations based on data supplied by tracking system 222. The keyboard 266 is configured to receive user input to those graphical user interfaces to control one or more functions of the surgical system 200.

The processing circuit 260 includes a processor and memory device. The processor can be implemented as a general purpose processor, an application specific integrated circuit (ASIC), one or more field programmable gate arrays (FPGAs), a group of processing components, or other suitable electronic processing components. The memory device (e.g., memory, memory unit, storage device, etc.) is one or more devices (e.g., RAM, ROM, Flash memory, hard disk storage, etc.) for storing data and/or computer code for completing or facilitating the various processes and functions described in the present application. The memory device may be or include volatile memory or non-volatile memory. The memory device may include database components, object code components, script components, or any other type of information structure for supporting the various activities and information structures described in the present application. According to an exemplary embodiment, the memory device is communicably connected to the processor via the processing circuit 260 and includes computer code for executing (e.g., by the processing circuit 260 and/or processor) one or more processes described herein.

More particularly, processing circuit 260 is configured to facilitate the creation of a preoperative surgical plan prior to the surgical procedure. According to some embodiments, the preoperative surgical plan is developed utilizing a three-dimensional representation of a patient's anatomy, also referred to herein as a “virtual bone model.” A “virtual bone model” may include virtual representations of cartilage or other tissue in addition to bone. To obtain the virtual bone model, the processing circuit 260 receives imaging data of the patient's anatomy on which the surgical procedure is to be performed (e.g., femur 206, pelvis 12). The imaging data may be created using any suitable medical imaging technique to image the relevant anatomical feature, including computed tomography (CT), magnetic resonance imaging (MRI), and/or ultrasound. The imaging data is then segmented (i.e., the regions in the imaging corresponding to different anatomical features are distinguished) to obtain the virtual bone model. For example, as described in further detail below, MRI-based scan data of a hip can be segmented to distinguish the femur from surrounding ligaments, cartilage, previously-implanted prosthetic components, and other tissue to obtain a three-dimensional model of the imaged hip.

Alternatively, the virtual bone model may be obtained by selecting a three-dimensional model from a database or library of bone models. In one embodiment, the user may use input/output device 262 to select an appropriate model. In another embodiment, the processing circuit 260 may execute stored instructions to select an appropriate model based on images or other information provided about the patient. The selected bone model(s) from the database can then be deformed based on specific patient characteristics, creating a virtual bone model for use in surgical planning and implementation as described herein.

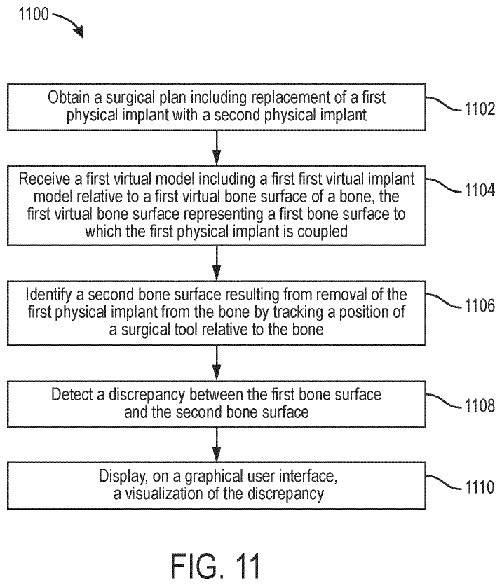

A preoperative surgical plan can then be created based on the virtual bone model. The surgical plan may be automatically generated by the processing circuit 260, input by a user via input/output device 262, or some combination of the two (e.g., the processing circuit 260 limits some features of user-created plans, generates a plan that a user can modify, etc.). In some embodiments, as described in detail below, the surgical plan may be generated and/or modified based on distraction force measurements collected intraoperatively. In some embodiments, the surgical plan may be modified based on qualitative intra-operational assessment of implant fixation (i.e., loose or fixed) and/or intra-operative bone defect mapping after primary implant removal, for example as described in detail below with reference to FIGS. 11-24.

The preoperative surgical plan includes the desired cuts, holes, surfaces, burrs, or other modifications to a patient's anatomy to be made using the surgical system 200. For example, for a total knee arthroscopy procedure, the preoperative plan may include the cuts necessary to form, on a femur, a distal surface, a posterior chamfer surface, a posterior surface, an anterior surface, and an anterior chamfer surface in relative orientations and positions suitable to be mated to corresponding surfaces of the prosthetic to be joined to the femur during the surgical procedure, as well as cuts necessary to form, on the tibia, surface(s) suitable to mate to the prosthetic to be joined to the tibia during the surgical procedure. As another example, in a hip arthroplasty procedure, the surgical plan may include the burr necessary to form one or more surfaces on the acetabular region of the pelvis 12 to receive a cup 28(a) and, in suitable cases, an implant augment. As yet another example, in a revision hip arthroplasty procedure, the surgical plan may include removal of an existing implant (i.e., a primary cup) and replacement of the existing implant with a replacement implant (i.e., a revision cup). Accordingly, the processing circuit 260 may receive, access, and/or store a model of the prosthetic to facilitate the generation of surgical plans.

The processing circuit 260 is further configured to generate a control object for the robotic device 220 in accordance with the surgical plan. The control object may take various forms according to the various types of possible robotic devices (e.g., haptic, autonomous, etc.). For example, in some embodiments, the control object defines instructions for the robotic device to control the robotic device to move within the control object (i.e., to autonomously make one or more cuts of the surgical plan guided by feedback from the tracking system 222). In some embodiments, the control object includes a visualization of the surgical plan and the robotic device on the display 264 to facilitate surgical navigation and help guide a surgeon to follow the surgical plan (e.g., without active control or force feedback of the robotic device). In embodiments where the robotic device 220 is a haptic device, the control object may be a haptic object as described in the following paragraphs.

In an embodiment where the robotic device 220 is a haptic device, the processing circuit 260 is further configured to generate one or more haptic objects based on the preoperative surgical plan to assist the surgeon during implementation of the surgical plan by enabling constraint of the surgical tool 234 during the surgical procedure. A haptic object may be formed in one, two, or three dimensions. For example, a haptic object can be a line, a plane, or a three-dimensional volume. A haptic object may be curved with curved surfaces and/or have flat surfaces, and can be any shape, for example a funnel shape. Haptic objects can be created to represent a variety of desired outcomes for movement of the surgical tool 234 during the surgical procedure. One or more of the boundaries of a three-dimensional haptic object may represent one or more modifications, such as cuts, to be created on the surface of a bone. A planar haptic object may represent a modification, such as a cut, to be created on the surface of a bone. A curved haptic object may represent a resulting surface of a bone as modified to receive a cup 28a and/or implant augment.

In an embodiment where the robotic device 220 is a haptic device, the processing circuit 260 is further configured to generate a virtual tool representation of the surgical tool 234. The virtual tool includes one or more haptic interaction points (HIPs), which represent and are associated with locations on the physical surgical tool 234. In an embodiment in which the surgical tool 234 is a spherical burr (e.g., as shown in FIG. 2), a HIP may represent the center of the spherical burr. If the surgical tool 234 is an irregular shape, for example as for a sagittal saw, the virtual representation of the sagittal saw may include numerous HIPs. Using multiple HIPs to generate haptic forces (e.g. positive force feedback or resistance to movement) on a surgical tool is described in U.S. application Ser. No. 13/339,369, titled “System and Method for Providing Substantially Stable Haptics,” filed Dec. 28, 2011, and hereby incorporated by reference herein in its entirety. In one embodiment of the present invention, a virtual tool representing a sagittal saw includes eleven HIPs. As used herein, references to a “HIP” are deemed to also include references to “one or more HIPs.” As described below, relationships between HIPs and haptic objects enable the surgical system 200 to constrain the surgical tool 234.

Prior to performance of the surgical procedure, the patient's anatomy (e.g., femur 206) is registered to the virtual bone model of the patient's anatomy by any known registration technique. One possible registration technique is point-based registration, as described in U.S. Pat. No. 8,010,180, titled “Haptic Guidance System and Method,” granted Aug. 30, 2011, and hereby incorporated by reference herein in its entirety. Alternatively, registration may be accomplished by 2D/3D registration utilizing a hand-held radiographic imaging device, as described in U.S. application Ser. No. 13/562,163, titled “Radiographic Imaging Device,” filed Jul. 30, 2012, and hereby incorporated by reference herein in its entirety. Registration also includes registration of the surgical tool 234 to a virtual tool representation of the surgical tool 234, so that the surgical system 200 can determine and monitor the pose of the surgical tool 234 relative to the patient (i.e., to femur 206). Registration allows for accurate navigation, control, and/or force feedback during the surgical procedure. Additional details relating to registration for hip arthroplasty procedures in some embodiments are described in detail below.

The processing circuit 260 is configured to monitor the virtual positions of the virtual tool representation, the virtual bone model, and the control object (e.g., virtual haptic objects) corresponding to the real-world positions of the patient's bone (e.g., femur 206), the surgical tool 234, and one or more lines, planes, or three-dimensional spaces defined by forces created by the robotic device 220. For example, if the patient's anatomy moves during the surgical procedure as tracked by the tracking system 222, the processing circuit 260 correspondingly moves the virtual bone model. The virtual bone model therefore corresponds to, or is associated with, the patient's actual (i.e. physical) anatomy and the position and orientation of that anatomy in real/physical space. Similarly, any haptic objects, control objects, or other planned automated robotic device motions created during surgical planning that are linked to cuts, modifications, etc. to be made to that anatomy also move in correspondence with the patient's anatomy. In some embodiments, the surgical system 200 includes a clamp or brace to substantially immobilize the femur 206 to minimize the need to track and process motion of the femur 206.

For embodiments where the robotic device 220 is a haptic device, the surgical system 200 is configured to constrain the surgical tool 234 based on relationships between HIPs and haptic objects. That is, when the processing circuit 260 uses data supplied by tracking system 222 to detect that a user is manipulating the surgical tool 234 to bring a HIP in virtual contact with a haptic object, the processing circuit 260 generates a control signal to the robotic arm 232 to provide haptic feedback (e.g., a force, a vibration) to the user to communicate a constraint on the movement of the surgical tool 234. In general, the term “constrain,” as used herein, is used to describe a tendency to restrict movement. However, the form of constraint imposed on the surgical tool 234 depends on the form of the relevant haptic object. A haptic object may be formed in any desirable shape or configuration. As noted above, three exemplary embodiments include a line, plane, or three-dimensional volume. In one embodiment, the surgical tool 234 is constrained because a HIP of the surgical tool 234 is restricted to movement along a linear haptic object. In another embodiment, the haptic object is a three-dimensional volume and the surgical tool 234 may be constrained by substantially preventing movement of the HIP outside of the volume enclosed by the walls of the three-dimensional haptic object. In another embodiment, the surgical tool 234 is constrained because a planar haptic object substantially prevents movement of the HIP outside of the plane and outside of the boundaries of the planar haptic object. For example, the processing circuit 260 can establish a planar haptic object corresponding to a planned planar distal cut needed to create a distal surface on the femur 206 in order to confine the surgical tool 234 substantially to the plane needed to carry out the planned distal cut.

For embodiments where the robotic device 220 is an autonomous device, the surgical system 200 is configured to autonomously move and operate the surgical tool 234 in accordance with the control object. For example, the control object may define areas relative to the femur 206 for which a cut should be made. In such a case, one or more motors, actuators, and/or other mechanisms of the robotic arm 232 and the surgical tool 234 are controllable to cause the surgical tool 234 to move and operate as necessary within the control object to make a planned cut, for example using tracking data from the tracking system 222 to allow for closed-loop control.

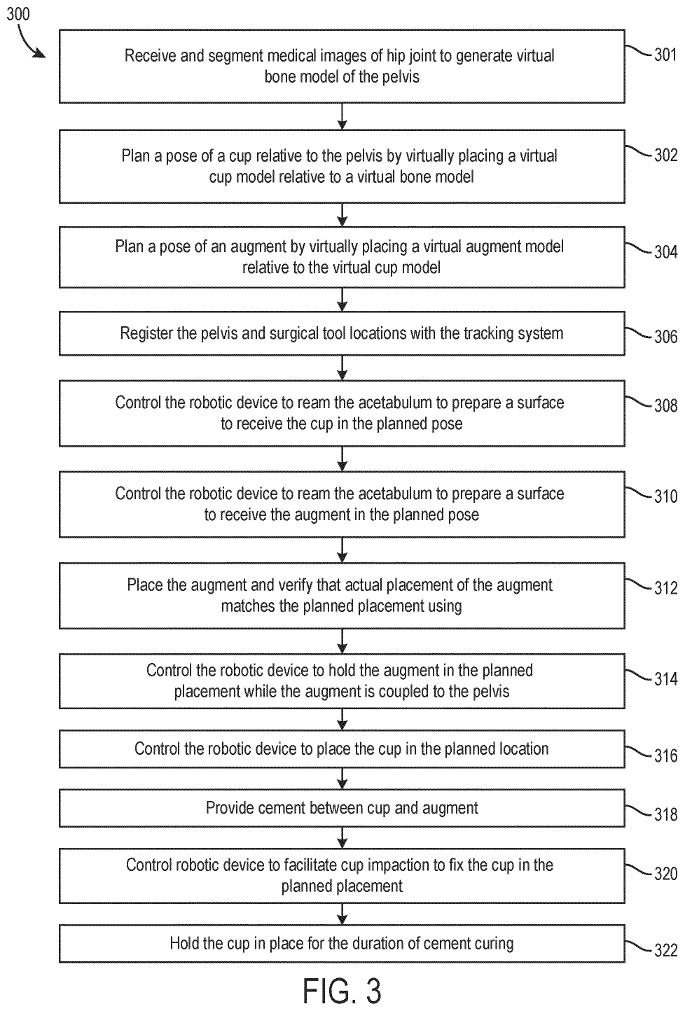

Referring now to FIG. 3, a flowchart of a process 300 for planning and conducting a hip arthroplasty procedure is shown, according to an exemplary embodiment. Process 300 can be executed by the surgical system 200 of FIG. 2. Additionally, FIGS. 4-30 show various systems, methods, graphical user interfaces, etc. used in process 300. Reference is made thereto to facilitate explanation of process 300. It should be understood that process 300 is not limited to the examples of FIGS. 4-30. Additionally, although FIGS. 3-30 illustrate embodiments of process 300 for planning and conducting a procedure relating to a hip, other embodiments are possible for planning and conducting procedures relating to other anatomy, for example shoulders or knees.

At step 301, medical images of the hip joint are received and segmented to generate a virtual bone model of the pelvis. For example, the medical images may be collected using CT technology, MRI technology, or some other medical imaging modality. The images are then segmented, i.e., processed to differentiate areas of the images that correspond to the pelvis, the femur, soft tissue, and/or one or more previously-implanted prosthetic components.

In revision hip arthroplasty cases (i.e., where a previously-implanted cup is shown in the images), a determination may be made of whether the previously-implanted cup is “fixed” (i.e., substantially rigidly coupled to the pelvis) or “loose” (i.e., at least partially detached from the pelvis”). If the previously-implanted cup is fixed, the shape, position, etc. of the previously-implanted cup may be determined and included in the virtual bone model of the pelvis, for example to facilitate registration at step 306 as described in detail below. If the previously-implanted cup is loose, the previously-implanted cup may be segmented out such that the loose cup is not included in the virtual bone model of the pelvis. Additionally, various corrections may be introduced to address distortions in CT or other imagery that may be caused by the materials of the previously-implanted cup and/or movement of a loose cup during imaging.

In some embodiments, step 301 is achieved automatically by the processing circuit 260 or other computing resource. In other embodiments, human input is used in cooperation with automated functions to achieve the segmentation and model generation of step 301.

At step 302, placement of an implant cup relative to the pelvis is planned by virtually placing a virtual cup model relative to a virtual bone model, i.e., relative to the virtual model of the pelvis generated at step 301 and, in some cases relative to previously-implanted components (e.g., primary cup, fracture plates, compression screws, etc.). The virtual cup model is a virtual representation of the cup implant to be implanted into the patient during the surgical procedure. Various cup sizes, shapes, types, etc. may be possible, and a different virtual cup model available for each cup. The virtual cup model is placed to provide a desired center of rotation for the hip joint (e.g., relative to the pelvis, relative to a patient's other hip, etc.) and ensure a full range of motion. Various software planning tools may be provided via the surgical system 200 to facilitate a surgeon or other user in selecting and evaluating the pose of the virtual cup model.

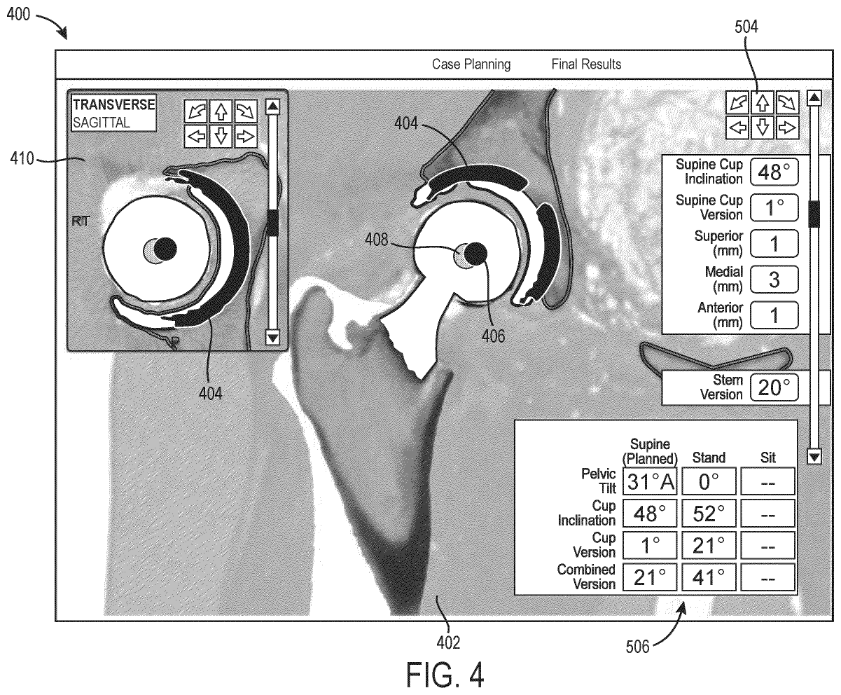

FIGS. 4-5 illustrate graphical user interfaces that can be generated by the processing circuit 260 and displayed on the display 264 to facilitate planning of cup placement at step 302. FIG. 4 shows a 2-dimensional visualization of a planned cup pose relative to CT images received at step 301. FIG. 5 shows a 3-dimensional visualization of the planned cup pose relative to a virtual bone model generated at step 301. Both are described in further detail below.

In FIG. 4, the graphical user interface 400 includes a first CT image 402 overlaid with a representation of the virtual implant cup 404. A center point (center of rotation) 406 of the virtual implant cup 404 is also shown. Additionally, as shown in FIG. 4, the graphical user interface 400 visualizes the previous center point 408 of the joint as imaged, i.e., before the surgical operation. In the example of FIG. 4, the graphical user interface 400 also shows a second CT image 410 (e.g., taken in a different plane) which is also overlaid with the virtual implant cup 404, the center point 406, and the previous center point 408. Advantageously, bone density information may be visible in the CT images 402, 410. The graphical user interface 400 may thereby facilitate a surgeon in determining placement of the virtual implant cup 404 relative to the imaged bones at step 302.

In FIG. 5, the graphical user interface 400 includes a 3-dimensional visualization of the virtual bone model 502 and of the virtual implant cup 404 placed relative to the virtual bone model 502. The graphical user interface 400 includes a previous center point 408 indicating a center of rotation of the hip joint as determined from the images as well as a center point 406 of the virtual implant cup 404. The graphical user interface 400 thereby facilitates a surgeon in viewing and adjusting the planned pose of the virtual implant cup 404.

As shown in FIGS. 4-5, the graphical user interface 400 includes control arrows 504 that can be selected to translate or rotate the virtual implant cup 404 relative to the virtual bone model 502. The graphical user interface 400 also includes data fields 506 that show various information that may be of interest to the user, for example, pelvic tilt, cup inclination, cup version, stem version, combined version, and superior, medial, and anterior distances. The graphical user interface 400 of FIGS. 4-5 thereby facilitates planning of implant cup placement relative to the pelvis at step 302.

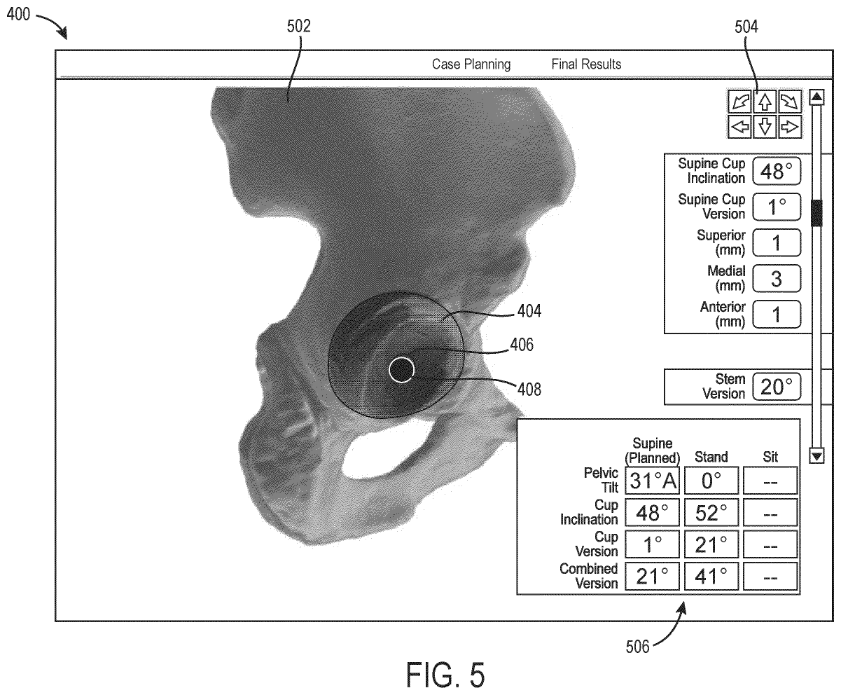

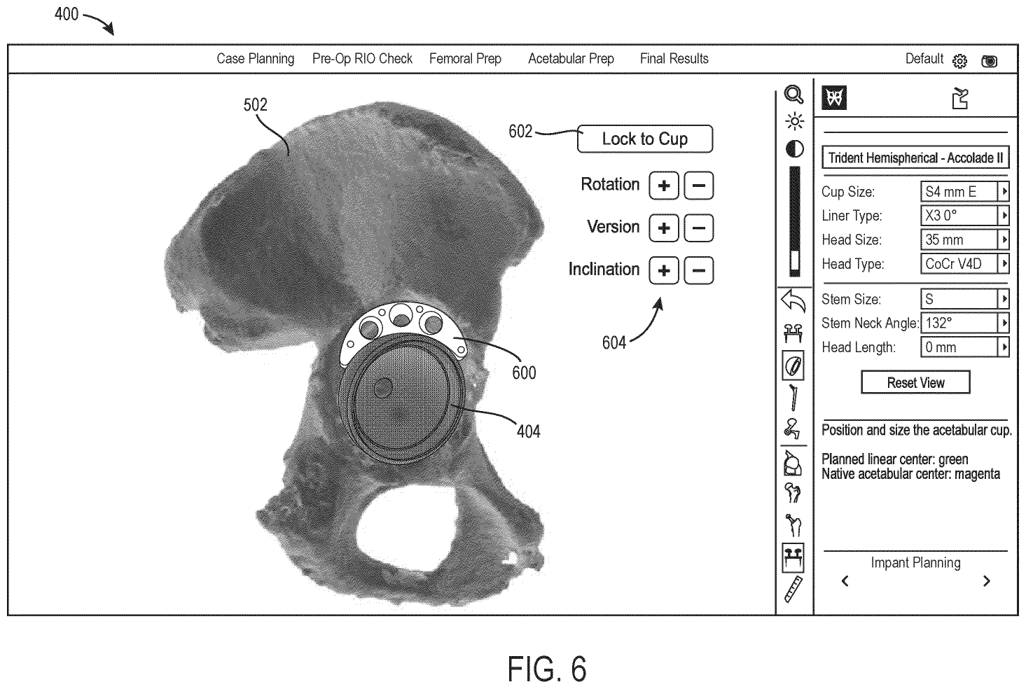

At step 304, placement of an implant augment is planned by virtually placing a virtual augment model relative to the virtual implant cup. For example, a determination may be made based on the visualization of the virtual bone model 502 of FIG. 5 or the CT images of FIG. 4 that an augment may be needed to reliably and securely install the implant cup in the position planed in step 302. An option can be selected via the graphical user interface 400 to include an augment. FIGS. 6-7 show views of the graphical user interface 400 that show a virtual augment model 600 and which facilitate selection of a desired placement of the virtual augment model 600. As shown in FIG. 6, the virtual augment 600 is visualized in a position relative to the virtual bone model 502 and the virtual implant cup 404 in a 3-D opaque view. As shown in FIG. 7, the virtual augment 600 is visualized in a position relative to the virtual bone model 502 in a translucent view and in two CT image views. FIGS. 6-7 are described in further detail below.

The graphical user interface 400 may include a warning message to indicate that an orientation of the virtual augment 600 violates a rule. For example, the rule may include an acceptable range of orientations of an augment required to support cutting a bone during a hip arthroplasty procedure. Step 304 can include comparing the orientation of the virtual augment 600 to the acceptable range of orientations to determine whether the virtual augment is oriented within the acceptable range of orientations and generating the warning if the virtual augmented is oriented outside the acceptable range of orientations. In some such examples, the graphical user interface 400 may include the warning message to indicate that the virtual augment 600 is improperly oriented (e.g., “upside down,” etc.). In some embodiments, the computing system 224 may allow planning the placement of the implant augment to proceed, despite the warning message being displayed on the graphical user interface 400. In some embodiments, steps of process 300 relating to bone preparation are omitted, prevented, abstained from, etc. in scenarios where such a warning is triggered (e.g., to allow planning but not robotically-assisted bone cutting for a “flying buttress” use of an augment).

In most cases, an implant augment has an interior surface that substantially matches an exterior surface of the implant cup, for example having a degree of curvature or radius substantially equal to the exterior surface of the implant cup. The augment is thereby configured to be placed adjacent to the implant cup and to provide structural support for the implant cup.

As shown in FIG. 6, the graphical user interface 400 includes a lock-to-cup button 602. When the lock-to-cup button 602 is selected, the virtual augment 600 is restricted to a pre-defined spacing relative to the virtual cup 404. For example, the virtual augment 600 may be positioned such that the virtual augment 600 is approximately two millimeters from the virtual cup 404. This spacing provides a volume which may be filled with cement or other adhesive during the procedure to couple the augment to the cup. As shown in FIG. 6, the graphical user interface 400 includes an array of control buttons 604 that can be selected to alter the rotation, version, and inclination of the virtual augment 600 while preserving the pre-defined spacing relative to the virtual cup 404. Accordingly, step 304 may include restricting the planned placement of the implant augment to a pre-defined spacing relative to the planned position of the cup.

As shown in FIG. 7, the graphical user interface 400 shows a representation of the virtual augment 600 and the virtual bone model 502 without the virtual cup 404. As shown in FIG. 7, the graphical user interface 400 may facilitate a surgeon in evaluating the contribution of the virtual augment 600 to formation of a surface for receiving the cup. CT views 704 show two-dimensional views of the virtual augment 600 relative to CT images collected of the patient's hip. The CT images may show bone density, a previously-implant cup, other implant components (e.g., screws, plates, etc. used to treat traumatic injury), and/or other useful information. The graphical user interface 400 of FIGS. 6-7 thereby facilitate planning of the implant augment relative to the implant cup and the pelvis. The graphical user interface 400 may also facilitate planning of screw trajectories of the implant and the augment, so that such screw trajectories are considered/planned simultaneously. This may ensure that the augment and implant cup are positioned such that the screws will not interfere with one another or with any existing hardware (e.g., trauma screws/plates). The screw trajectories may also be visualized relative to bone density to ensure adequate screw fixation is achieved.

Steps 302 and 304 can thereby result in a planned pose of the implant cup and a planned pose of the implant augment. Such planning (i.e., steps 301-304) may occur pre-operatively and/or intraoperatively. The remaining steps of process 300 occur intraoperatively, i.e., during the surgical procedure.

At step 306, a registration process is executed to register the relative positions of the patient's pelvis, the surgical tool(s), the robotic device, and/or other tracked probes or instruments. For example, a probe may be tracked by the tracking system 222 and touched to various points on the pelvis to determine a pose of the pelvis. Various registration methods are described above with reference to FIG. 2.

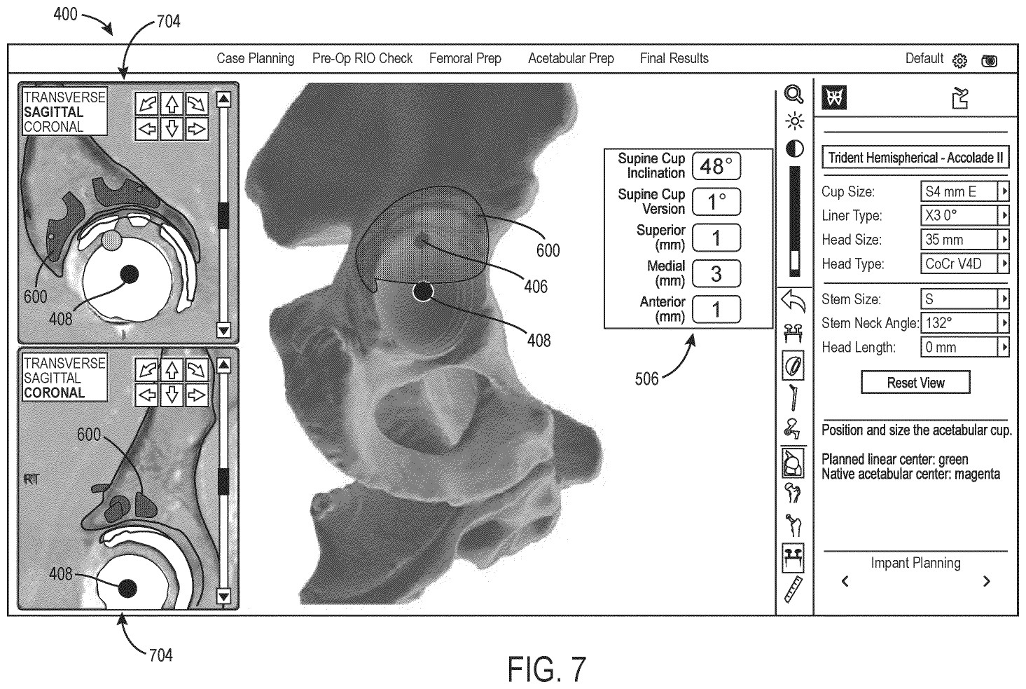

In the case of revision hip arthroplasty procedures, different registration workflows may be used depending on whether the previously-implanted cup is loose or fixed. FIG. 8 shows a flowchart of a process 800 for registration in revision hip arthroplasty procedures, according to an exemplary embodiment.

As illustrated in FIG. 8, if the previous cup is fixed, the liner of the previous implant (i.e., implanted in a previous procedure) is removed at step 802. At step 804, the location of the pelvis is registered via the previous implant cup, which is fixed to the pelvis. For example, a tracked probe can be touched to various locations on the previous implant cup to determine a pose of a surface of the previous implant cup. As another example, intra-operative imaging (e.g., x-ray) may be used to determine a pose of a surface of the previous implant cup. Because the geometric relationship between the previous implant cup and the pelvis is fixed and known from the medical images received at step 301, such data can be used for registration of the pelvis. A tracked probe and/or intraoperative imaging may also be used to locate and register existing hardware (e.g., trauma screws/plates) to facilitate avoidance of such structures during a procedure (e.g., by creating virtual control objects around the located positions of such structures). Following registration, the previous cup is removed at step 806 to allow the revision implant to be installed. In some embodiments, haptic guidance is used to facilitate removal of the previous (primary, existing) implant, for example as described in U.S. Patent Application 20180014891. For example, a virtual control object can be generated by referencing a library of implant designs to determine a geometry of the relevant implant, identifying the edges of the previous implant using a probe, and generating haptic boundaries based on the probed edges.

Also as illustrated in FIG. 8, if the previous cup is loose (i.e., not fixed), the previous cup and liner are removed at step 808 prior to registration of the pelvis. At step 810, the pelvis is registered without the previous cup. For example, a probe may be touched to various points around or in the region from which the previous cup was removed. FIGS. 11-24 illustrate additional processes and graphical user interfaces that may be used for mapping a bone surface (i.e., registering the pelvis) after a previous cup has been removed.

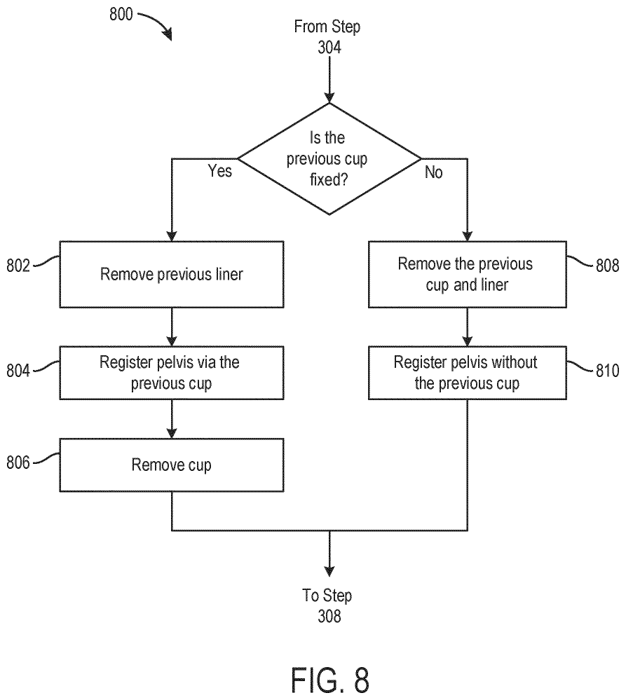

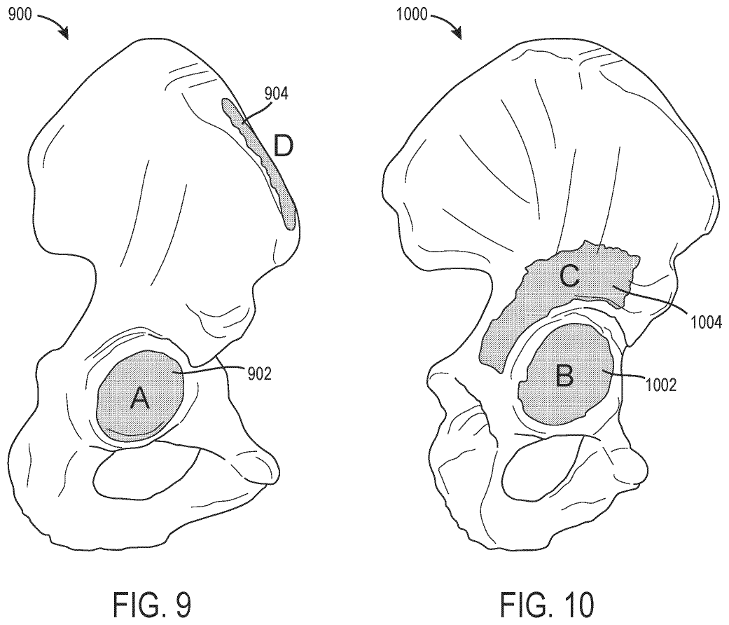

To further illustrate the registration of step 306 according to some embodiments, FIGS. 9-10 depict regions of the pelvis that may be used for registration in various scenarios. FIG. 9 illustrates a virtual bone model 900 that includes a fixed cup, while FIG. 10 illustrates a virtual bone model 1000 in which a loose cup has been removed, leaving an approximated, smooth surface. FIGS. 9-10 include demarcation of several registration regions, shown as region A 902, region B 1002, region C 1004, and region D 904.

In a scenario with a fixed cup, registration points (i.e., points touched by a tracked probe and used for registration) can be taken in region A 902, which corresponds to a surface of the previously-implanted fixed cup. Such points may be particularly reliable and accessible, as region A 902 is exposed during surgery to allow for removal of the previously-implanted cup. Other points may also be taken, for example in region D 904 (along the iliac crest) and/or region C 904 (above the acetabulum).

In a scenario with a loose cup, registration points can be taken in region B 1002, which corresponds to an acetabular surface exposed when the loose cup is removed from the patient. For example, registration points may be taken around a rim of region B 1002. The reliability of such points may be dependent on the accuracy of the segmentation of step 301 in differentiating the surface of the bone in the pre-operative imagery from the loose cup, which is removed to expose the surface of region B 1002. In some embodiments, registration of the pelvis is achieved in the loose cup scenario without using acetabular registration points (without using registration points in region A 902 or region B 1002) and by using extra-acetabular registration points (e.g., points in region C 1004 and/or region D 904).

Registration as conducted at step 306 thereby facilitates a mapping of the actual pose of the pelvis in real space to a virtual position of the virtual bone model 502 in virtual space. The virtually-planned poses of the virtual implant augment and the virtual implant cup can then also be associated with real poses in real space (e.g., relative to a coordinate system used by the tracking system 222).

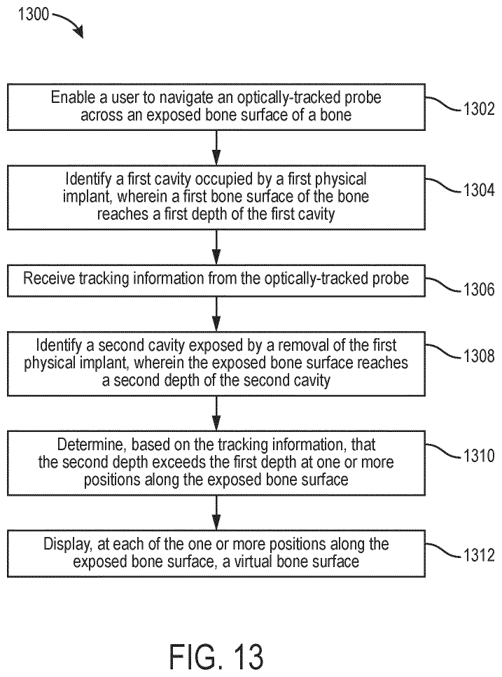

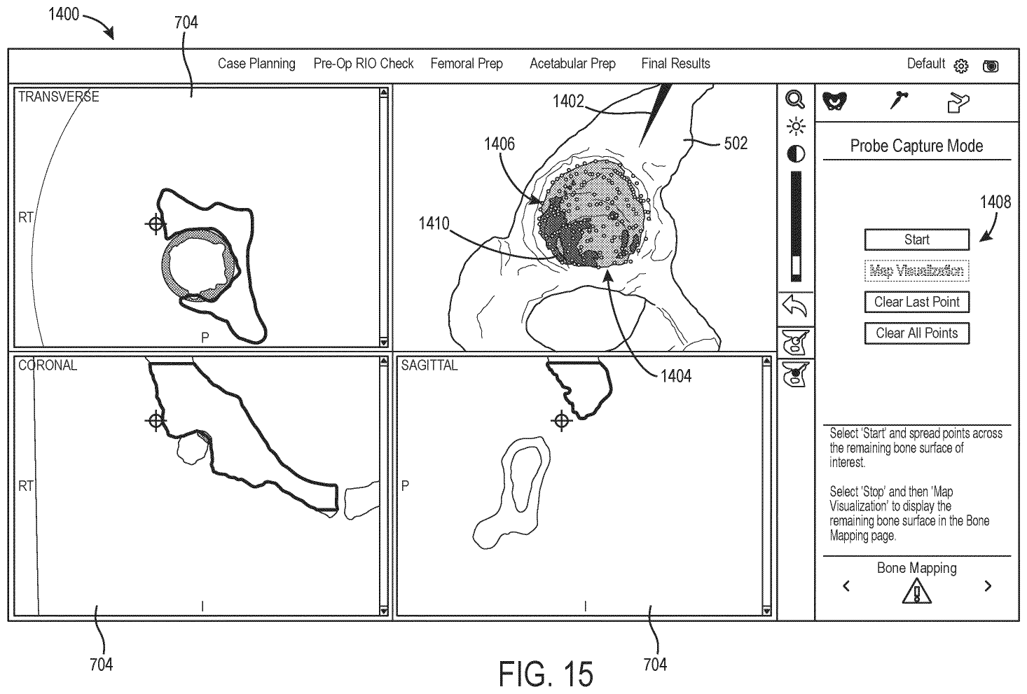

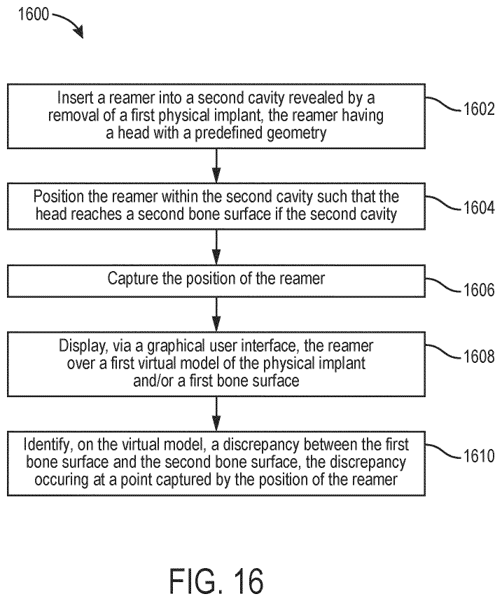

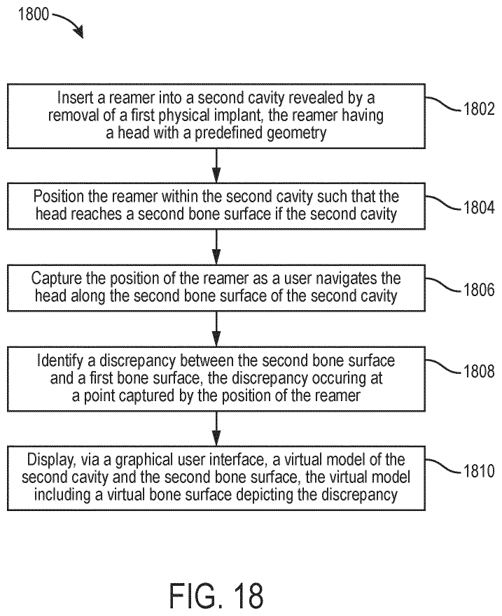

The primary cup (i.e., the existing implant, the first physical implant, etc.) can then be removed using standard techniques. In some cases, removal of the primary cup may result in an unexpected defect cavity (i.e., a second cavity) which was not accounted for by the original surgical plan. In such cases, the tracked probe may be used to define a contour (size, shape, pose, etc.) of the defect cavity, for example by tracking the location of the probe as the probe is touched to various positions on the surface of the defect cavity, traced/painted along the defect cavity, etc. For example, FIG. 13 illustrates a process 1300 for mapping a surface of the defect cavity using a probe, as described in greater detail below. Alternatively or additionally, a reamer may be used to define a contour (size, shape, pose, etc.) of the defect cavity, for example by tracking the location of the reamer as the reamer is touched to one or more positions on the surface of the defect cavity, traced/painted along the defect cavity, etc. For example, FIGS. 16 and 18 illustrates processes 1600 and 1800, respectively, for mapping a surface of the defect cavity using a reamer, as described in greater detail below. The virtual bone model may then be updated to include a virtual representation of the defect cavity, so that the virtual bone model substantially matches the actual form of the bone after primary cup removal. For example, FIGS. 15, 17, and 19-24 each depict a virtual bone model that has been updated to include a virtual representation of the defect cavity, as described in greater detail below. The surgical plan can then be adjusted/updated to account for the defect cavity, for example by modifying a size or pose of at least one of an implant cup, an augment, or other implant component. Intra-operative registration and bone model updates can also be used to correct for voids from a segmentation process or clarify regions of scatter in the original imaging (e.g., CT images).

Although reference is made to implementing the various features described herein within the specific context of a revision hip arthroplasty procedure (i.e., a procedure during which an existing implant is removed and replaced with a new implant), it should be appreciated that the features described herein may be incorporated into a variety of clinical scenarios. For example, the features described herein may also be introduced during a primary hip arthroplasty procedure (i.e., a procedure during which a hip receives an implant for the first time), during a removal of a mass (i.e., a procedure during which an unwanted mass such as a tumor is removed from a bone surface or other anatomical structure), during an assessment of a bone surface where the bone has a pre-existing defect, and so on. The features described herein may relate to these and still other clinical scenarios during which a bone surface is analyzed and/or otherwise relevant to the procedure.

As illustrated by FIG. 11, process 1100 for mapping a bone surface of a defect cavity during the process of FIG. 3 is shown. The defect cavity refers to a cavity that is exposed by the removal of the primary cup during a revision hip arthroplasty procedure. The process 1100 begins by obtaining a surgical plan that includes replacing a first physical implant with a second physical implant at step 1102. The first physical implant refers to the primary cup (i.e., a previously-implanted cup) that is being replaced during the revision hip arthroplasty procedure. The second physical implant refers to a revision implant (i.e., a replacement cup) that is replacing the primary cup during the revision hip arthroplasty procedure. For example, the first physical implant may be depicted by region A 902 in FIG. 9 (i.e., which corresponds to a surface of a previously-implanted fixed cup). As described above, region A 902 is exposed during surgery to allow for removal of the previously-implanted cup (i.e., the first physical implant). The surgical plan obtained during step 1102 may be generated by the processing circuit 260, as described above with reference to FIG. 2. Process 1100 continues with receiving a first virtual model including a first virtual implant model relative to a first virtual bone surface of a bone at step 1104. The first virtual implant model refers to a virtual model representing the first physical implant (i.e., the primary cup) being replaced during the revision hip arthroplasty procedure. The first virtual bone surface represents a first bone surface to which the first physical implant is coupled. Results of medical imaging (e.g., CT images) can be segmented (e.g., via automated image processing) to distinguish the first virtual implant model from the first virtual bone surface based on pre-operative or intra-operative imagining of the patient. In some embodiments, the virtual model may be depicted by a graphical user interface on the display 264. For example, the first virtual model may include the virtual implant cup 404 relative to the virtual bone model 502, as depicted by graphical user interface 400, where the virtual implant cup 404 is a virtual representation of the primary cup being replaced during the revision hip arthroplasty procedure.

As shown in FIG. 11, step 1106 includes identifying a second bone surface resulting from removal of the first physical implant from the bone by tracking a position of a surgical tool relative to the bone. That is, removal of the first physical implant (i.e., the primary cup) exposes a second bone surface that is distinct from the first bone surface to which the first physical implant was coupled. The second bone surface may be distinct due to defects caused by excess bone being inadvertently extracted during the removal of the first physical implant, due to other bone defects, due to limitations of pre-operative imagining and modeling techniques, etc. For example, the second bone surface may be depicted by region B 1002, which corresponds to the acetabular surface exposed when a loose cup is removed from the patient, as described above.

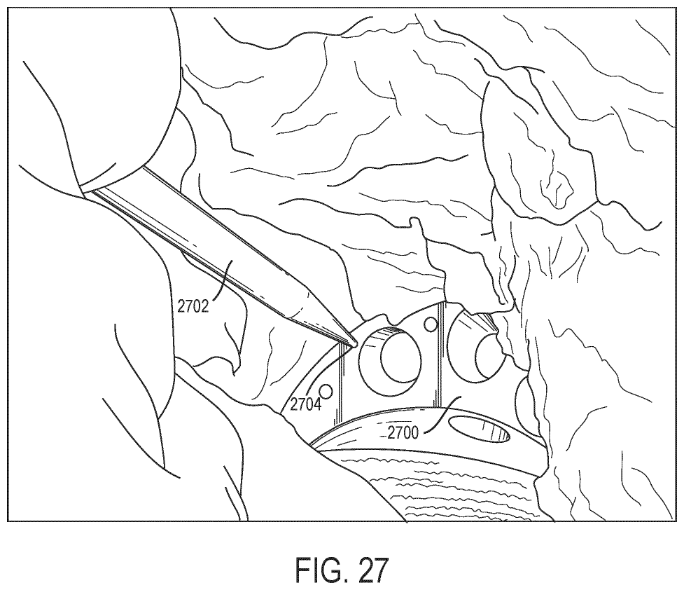

In some embodiments, the surgical tool may be the surgical tool 234 coupled to the robotic arm 232. In such embodiments, the surgical tool 234 may be tracked by the tracking system 222, such that as the surgical tool 234 contacts the second bone surface, the tracking system 222 may register (i.e., map) the second bone surface (i.e., the bone surface exposed upon the removal of the primary cup). In some embodiments, the surgical tool 234 is a probe, as described by the process and interfaces illustrated in FIGS. 13-15. In other embodiments, the surgical tool 234 is a reamer, as described by the processes and interfaces illustrated in FIGS. 16-24. FIG. 12 depicts a detailed view of parts of the surgical system of FIG. 2 used to perform the process of FIG. 11. For example, as shown in FIG. 12, the surgical tool 234 used to track the second bone surface during step 1106 of process 1100 may include a reamer held by the robotic arm 232 of the robotic device 220. Alternatively or additionally, the surgical tool 234 may include a probe (i.e., probe 268, as described above, navigation probe 2702, as described below with reference to FIG. 27) held by the robotic arm 232 of the robotic device 220.

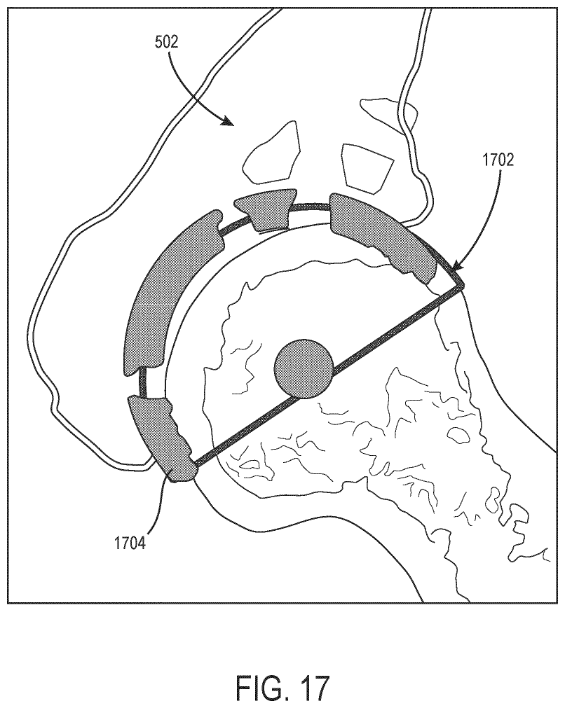

After identifying the second bone surface at step 1106, process 1100 continues by detecting a discrepancy between the first virtual bone surface (i.e., the virtual representation of the bone surface to which the first physical implant/primary cup was previously coupled; a surface of a virtual bone model interfacing with a virtual primary implant model as created from pre-operative imaging) and the second bone surface (i.e., the bone surface exposed after the removal of the first physical implant/primary cup) at step 1108. The discrepancy may be detected using an optically-tracked probe, as described below with reference to FIGS. 13-15, a reamer in a single-capture mode, as described below with reference to FIGS. 16-17, and/or a reamer in a multi-capture mode, as described below with reference to FIGS. 18-24. After detecting the discrepancy at step 1108, the process 1100 includes displaying a visualization of the discrepancy on a graphical user interface at step 1110. In some embodiments, the visualization of the discrepancy may include a mapping of the second bone surface compared to the first bone surface. For example, a discrepancy between the second bone surface and the first bone surface may be illustrated using a unique coloration or other visualization technique, as shown in FIGS. 15, 17, and 19-24.

FIG. 13 illustrates a process 1300 for mapping a bone surface of a defect cavity using a probe during the process of FIG. 11, according to an exemplary embodiment. For example, the probe may be the surgical tool 234 with which the bone surface of the defect cavity is mapped. In some embodiments, the probe may be the probe 268, as shown in FIG. 2. As shown in FIG. 13, process 1300 begins by enabling a user to navigate an optically-tracked probe across an exposed bone surface of a bone. That is, process 1300 begins once the primary cup (i.e., the existing explant) is removed and the bone surface is exposed. The optically-tracked probe may be tracked using any of the methods and/or techniques described herein, for example, using the tracking system 222, as described above with reference to FIG. 2. Navigating the optically-tracked probe may include contacting the exposed bone surface using a tip of the optically-tracked probe such that the tip makes contact with an entirety of the exposed bone surface (i.e., the user is enabled to “paint” the exposed bone surface using the tip of the optically-tracked probe).

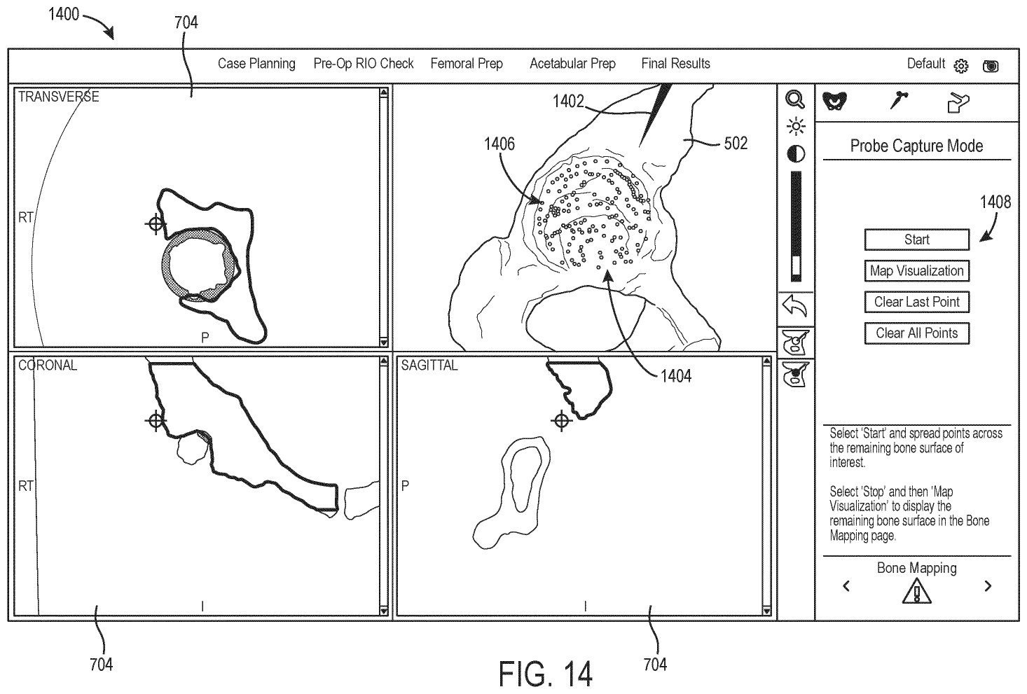

Process 1300 continues with identifying a first cavity occupied by a first physical implant (i.e., the primary cup) at step 1304, where a first bone surface of the bone reaches a first depth of the first cavity. In some embodiments, the first cavity and the first depth of the first cavity may be identified from CT images of a patent's bone prior to the primary implant being inserted. That is, the first cavity is a cavity occupied by the first physical implant. Therefore, the first bone surface refers to the bone surface to which the first physical implant is coupled. In some embodiments, the first bone surface may be depicted on a graphical user interface during step 1304. For example, FIGS. 14-15 depict the virtual bone model 502 on graphical user interface 1400, where the virtual bone model 502 includes a virtual representation of the first bone surface that defines the first cavity (i.e., represented by the virtual cavity 1406). FIGS. 14-15 are explained in further detail below.

As illustrated in FIG. 13, process 1300 includes receiving tracking information from the optically-tracked probe at step 1306. That is, the tracking information may be received as the user navigates (physically moves) the optically-tracked probe across the exposed bone surface of the bone, as described above with reference to step 1302 of process 1300. In some embodiments, the tracking information includes the positions of the optically-tracked probe relative to a bone model (i.e., the virtual bone model 502). For example, the positions of the optically-tracked probe may be depicted by the tracked points 1408 illustrated across the virtual cavity 1406 of graphical user interface 1400, as described in greater detail below. The positions of the optically-tracked probe can be captured continuously, periodically (e.g., one point collected per quarter second, per half second, or per second, etc.), or on demand by the user (e.g., responsive to an input to a foot pedal, to a button on the probe, or to an icon on a graphical user interface).

Based on the tracking information received at step 1306, process 1300 continues by identifying a second cavity exposed by the removal of the first physical implant at step 1308, where the exposed bone surface reaches a second depth of the second cavity. The second cavity refers to the defect cavity revealed after the primary cup is removed during a revision hip arthroplasty procedure, which is therefore defined by the exposed bone surface. That is, the virtual cavity 1406 may represent a cavity that exists prior to the removal of the primary cup (i.e., defined by the virtual bone model 502), while the second cavity may be defined by an actual cavity (i.e., existing in real space) that is exposed after the removal of the primary cup.

Thus, as the user navigates the optically-tracked probe across the exposed bone surface, and as the tracking information is received at step 1306, there may be one or more discrepancies detected between the first bone surface (i.e., depicted by the virtual bone model 502) and the exposed bone surface. In some embodiments, the one or more discrepancies are positions along the exposed bone surface where the exposed bone surface reaches a depth of the defect cavity (i.e., the second depth of the second cavity) that is distinct from the first depth of the first cavity previously occupied by the primary cup (i.e., as depicted by the virtual bone model 502). The one or more discrepancies may be detected at step 1310 of process 1300, during which the second depth is determined to exceed the first depth at one or more positions along the exposed bone surface. In other words, the one or more discrepancies reveal positions along the exposed bone surface relative to the virtual bone model 502 where excess bone may have been inadvertently removed with the removal of the primary cup. At each of the one or more positions along the exposed bone surface where the second depth of the second cavity is determined to exceed the first depth of the first cavity, a virtual bone surface may be displayed during step 1312 of process 1300. For example, the virtual bone surface may be illustrated as the virtual bone surface 1410 on graphical user interface 1400, as described in greater detail below.

In some embodiments, the step 1306 can include classifying a plurality of points (i.e., probe tip positions) represented in the tracking data as being proud of the virtual bone model 502, substantially aligned with a surface of the virtual bone model 502, or at a depth into (e.g., within) the virtual bone model 502. For example, step 1306 can include checking, for each point, whether that point is inside, outside, or at a surface of the virtual bone model 502 (e.g., using a computed solid geometry representation of the virtual bone model 502 and comparing points to coordinates occupied by the virtual bone model 502). Points proud of the virtual bone model 502 (i.e., outside the virtual bone model 502) can be discarded as erroneous while points on the virtual bone model 502 can be treated as consistent with the first cavity and points inside the virtual bone model 502 can be treated as defining the second cavity.

FIG. 14 illustrates a graphical user interface 1400 that can be used with the process of FIG. 13, according to an exemplary embodiment. For example, the graphical user interface 1400 may be used during process 1300 to depict the tracking information received at step 1306 from the optically-tracked probe. The graphical user interface 1400 may include a virtual probe 1402 that represents the physical probe (i.e., the optically-tracked probe) being used to trace the exposed bone surface. In some embodiments, a position of the virtual probe 1402 relative to the virtual bone model 502 may mirror a position of the optically-tracked probe (i.e., the surgical tool 234) relative to the exposed bone surface of a patient's bone. That is, as the user (i.e., a surgeon performing a revision hip arthroplasty procedure) navigates the optically-tracked probe across the exposed bone surface, the graphical user interface 1400 depicts the virtual probe 1402 moving across the virtual bone model 502 in mirrored positions to those of the optically-tracked probe. The graphical user interface 1400 also depicts the virtual bone model 502 including a virtual cavity 1404. The virtual cavity 1404 is a virtual representation of the first cavity that is occupied by the first physical implant (i.e., the primary cup) prior to removal during the revision hip arthroplasty procedure. As shown in FIG. 14, the graphical user interface 1400 also includes the CT views 704.

In order to map (e.g., collect information about a contour of) the exposed bone surface, a user may click on the graphical user interface 1400 when the optically-tracked probe contacts a desired point along the exposed bone surface. After the user clicks on the graphical user interface 1400, a tracked point 1406 may be shown at a point on the virtual bone model 502 corresponding to the point on the exposed bone surface that is in contact with the optically-tracked probe when the user engages with the graphical user interface 1400. In some embodiments, the user may also cause the computing system 224 to map the exposed bone surface by pushing on a foot pedal coupled to the robotic device 220 when the optically-tracked probe contacts the desired point along the exposed bone surface. After the user engages with (i.e., pushes, clicks, presses, etc.) the foot pedal, a tracked point 1406 may be shown at a point on the virtual bone model 502 corresponding to the point on the exposed bone surface that is in contact with the optically-tracked probe when the user engages with the foot pedal.

Similarly, in some embodiments, the user may cause the computing system 224 to map the exposed bone surface using a button on the optically-tracked probe. For example, the optically-tracked probe may include a capture button and a deletion button. The capture button may be used to capture a point along the exposed bone surface where the optically-tracked probe is positioned, relative to the exposed bone surface, at the moment of capture. The deletion button may be used to delete a most recent point that was captured along the exposed bone surface. After the user engages with the capture button, the graphical user interface 1400 may display a tracked point 1406 at a point on the virtual bone model 502 corresponding to the point on the exposed bone surface that is in contact with the optically-tracked probe when the user clicks the capture button. Similarly, after the user engages with the deletion button, the graphical user interface 1400 may remove a tracked point 1406 from a point on the virtual bone model 502 corresponding to the point on the exposed bone surface that was most recently captured. In some embodiments, if the user holds down the capture button, points will be captured continuously (e.g., at a particular frequency) as the user moves the probe along the exposed bone surface.

As shown in FIG. 14, the graphical user interface may also include selectable elements 1408. In some embodiments, the selectable elements 1408 may include a “Start” button, a “Map Visualization” button, a “Clear Last Point Button,” and a “Clear All Points” button. The “Start” button may be used to initiate process 1300 and enable the user to navigate the optically tracked probe across the exposed bone surface of the bone. For example, after a user clicks on the “Start” button, the virtual probe 1402 may be displayed on the graphical user interface 1400 and the user may cause the computing system 224 to begin to capture the tracked points 1406 using one of the methods/techniques described herein. In some embodiments, once a user clicks on the “Start” button, the graphical user interface 1400 may display a “Stop” button in place of the “Start” button among the selectable elements 1408. In such embodiments, the “Stop” button may end the mapping process and may prevent the computing system 224 from capturing any additional points along the exposed bone surface.

The “Map Visualization” button may be used to generate a virtual bone surface displayed on the graphical user interface 1400. For example, the virtual bone surface may refer to the virtual bone surface that is displayed during step 1312 of process 1300. That is, after a user clicks on the “Map Visualization” button, the graphical user interface 1400 may display a visualization of the defect cavity relative to the virtual cavity 1404 of the virtual bone model 502. In some embodiments, the visualization of the defect cavity may be the virtual bone surface 1410, as described below with reference to FIG. 15.

The “Clear Last Point” button may perform a functionality similar to that of the deletion button on the optically-tracked probe described above. Namely, the “Clear Last Point” button may be used to delete a most recent point that was captured along the exposed bone surface. After a user selects the “Clear Last Point” button, the graphical user interface 1400 may remove a tracked point 1406 from the virtual bone model 502 corresponding to the tracked point 1406 that was most recently captured. Similarly, the “Clear All Points” button may be used to delete all tracked points 1406 from the graphical user interface 1400. For example, the “Clear All Points” button may instruct the computing system 224 to restart (i.e., start over) the mapping process described herein.

As illustrated in FIG. 15, after a user clicks on the “Map Visualization” button, the graphical user interface 1400 may display a virtual bone surface 1410 (i.e., the second virtual bone surface, the exposed virtual bone surface, etc.) that virtually represents the bone surface that is revealed after removal of an existing implant (i.e., the first physical implant, the primary cup, etc.). The virtual bone surface 1410 may therefore define a virtual representation of the defect cavity that is exposed after the removal of the existing implant during a revision hip arthroplasty procedure. In some embodiments, the virtual bone surface 1410 may be depicted with the tracked points 1406 over the virtual cavity 1404 of the virtual bone model 502. A unique coloration or other visualization technique may be used to generate the virtual bone surface 1410 such that the virtual bone surface 1410 identifies points along the exposed bone surface (i.e., the second bone surface of the second cavity) that reach a greater depth (i.e., a second depth) than the corresponding points along the first bone surface of the cavity occupied by the primary implant (i.e., the first cavity). For example, points/regions along the virtual bone model 502 depicted in a lighter shade may illustrate where the second depth of the second bone surface matches the first depth of the first bone surface (e.g., where points collected with the probe substantially align with the first bone surface), whereas points/regions along the virtual bone model 502 depicted in a darker shade may illustrate areas where the second depth of the second bone surface exceeds the first depth of the first bone surface.

Therefore, the virtual bone surface 1410 may provide a mapping of an actual bone surface of the defect cavity during a revision hip arthroplasty procedure such that the computing system 224 may update the surgical plan (i.e., the surgical plant received at step 1102 of process 1100) based on the virtual bone surface 1410 depicted on the graphical user interface 1400. For example, updating the surgical plan may include updating a shape or planned position of the replacement implant to account for the discrepancies detected in the defect cavity after the removal of the primary cup. As another example, updating the surgical plan may include updating the shape of and/or inserting an augment to account for the discrepancies detected in the defect cavity after the removal of the primary cup. In other words, updating the surgical plan may include updating a size, type, position, and/or orientation of at least one of an implant or an augment such that the updated implant or augment occupies (i.e., fills) the defect cavity (i.e., the second cavity).

FIG. 16 illustrates a process 1600 for mapping a bone surface of a defect cavity using a reamer during the process of FIG. 11, according to an exemplary embodiment. More specifically, process 1600 uses a single-capture mode to detect discrepancies during process 1100. In some embodiments, as shown in FIG. 12, the reamer may be the surgical tool 234 with which the bone surface of the defect cavity is mapped. FIG. 16 may begin by inserting a reamer into a second cavity revealed by a removal of a first physical implant. The second cavity refers to the defect cavity revealed by the removal of the primary cup, as described herein. The second cavity may also refer to the second cavity identified during step 1308 of process 1300, as described above.

The reamer inserted at step 1602 may have a head with a known geometry (e.g., hemispherical) such that the surgical system 200 has knowledge of the geometry (including dimensions) of the reamer being used during process 1600 (e.g., based on a user selection of which of a set of reamer heads is being used). In some embodiments, the reamer may be depicted as virtual reamer 1702, described in greater detail below. Because the surgical system 200 has the knowledge of the geometry of the reamer being used, the robotic device 220, in combination with the tracking system 222, provides tracking data to indicate where the tool center point of the reamer is relative to the patient's pelvis. Therefore, based on the tool center point and the reamer geometry (i.e., a radius of the reamer head), the surgical system 200 can determine where an outer surface of the reamer head is positioned relative to the bone.

As shown in FIG. 16, process 1600 continues by positioning the reamer within the second cavity such that the head reaches a second bone surface of the second cavity at step 1604. The second bone surface refers to a bone surface that is exposed upon the removal of the first physical implant. Therefore, the head reaches the second bone surface once the head reaches a depth of the second cavity. The second bone surface may be a bone surface of the defect cavity, as described herein. Once the reamer is positioned within the second cavity at step 1604, the position of the reamer is captured at step 1606. A user may cause the computing system 224 to capture the position of the reamer by clicking, pushing, or otherwise engaging with a foot pedal configured to capture a position of an optically-tracked object (i.e., the reamer) at the moment of engagement. Alternatively or additionally, a user may cause the computing system 224 to capture the position of the reamer by clicking on a graphical user interface when the head of the reamer reaches the second bone surface of the second cavity, or entering a voice command, pulling a trigger on an end effector of a robotic arm holding the reamer, or otherwise providing an input to the surgical system 200.

Step 1608 includes displaying, via a graphical user interface, the reamer over a first virtual model of the first physical implant and/or a first bone surface. For example, FIG. 17 depicts the virtual reamer 1702 over a virtual model including the virtual bone model 502 and a virtual implant 1704. In some embodiments, the virtual model depicted in FIG. 17 may be generated using CT images of an anatomical region of interest (i.e., a hip area) before the primary cup is removed. The virtual reamer 1702 represents the reamer that is inserted into the second cavity during step 1602 at the position captured during step 1606. The virtual implant 1704 refers to a virtual representation of the primary cup that is being replaced during the revision hip arthroplasty procedure. Therefore, the virtual bone model 502 and the virtual implant 1704 define the first bone surface by depicting a cavity (i.e., the first cavity) within the virtual bone model 502 that is occupied by the virtual implant 1704.

As shown in FIG. 16, process 1600 continues by identifying, on the virtual model displayed during step 1608 and depicted by FIG. 17, a discrepancy between the first bone surface and the second bone surface at step 1610. The discrepancy occurs at a point captured by the position of the reamer at step 1606. In some embodiments, the discrepancy refers to a point along the second bone surface (i.e., a point reached by the head of the reamer during step 1604) where the second cavity (i.e., the defect cavity) reaches a greater depth than a depth of the first cavity at a corresponding point along the first bone surface (e.g., a point or region where a virtual representation of the head of the reamer reached into the first virtual bone model). The identified discrepancy may correspond to a point along the first bone surface where excess bone was inadvertently removed with the removal of the primary cup during the revision hip arthroplasty procedure. Therefore, a depth of the second cavity (i.e., the defect cavity) may exceed a depth of the first cavity (i.e., the cavity occupied by the primary cup) where excess bone has been removed. For example, FIG. 17 illustrates where the depth of the second cavity exceeds the depth of the first cavity by gaps within the virtual implant 1704. That is, the virtual reamer 1702 is shown to reach a greater depth within the first cavity of the virtual bone model 502 than a depth within the first cavity reached by the first physical implant (i.e., the primary cup) at the points in the virtual model of FIG. 17 where the virtual implant 1704 is shown as being segmented.

FIG. 18 illustrates a process 1800 for mapping a bone surface of a defect cavity using a reamer during the process of FIG. 11, according to an exemplary embodiment. More specifically, process 1800 uses a multi-capture mode to detect discrepancies during process 1100. In some embodiments, as shown in FIG. 12, the reamer is the surgical tool 234 with which the bone surface of the defect cavity is mapped. FIG. 18 may begin by inserting a reamer into a second cavity revealed by a removal of a first physical implant. For example, the second cavity may refer to the defect cavity revealed by the removal of the primary cup, as described herein. The second cavity may also refer to the second cavity identified during step 1308 of process 1300 and during step 1602 of process 1600, as described above.

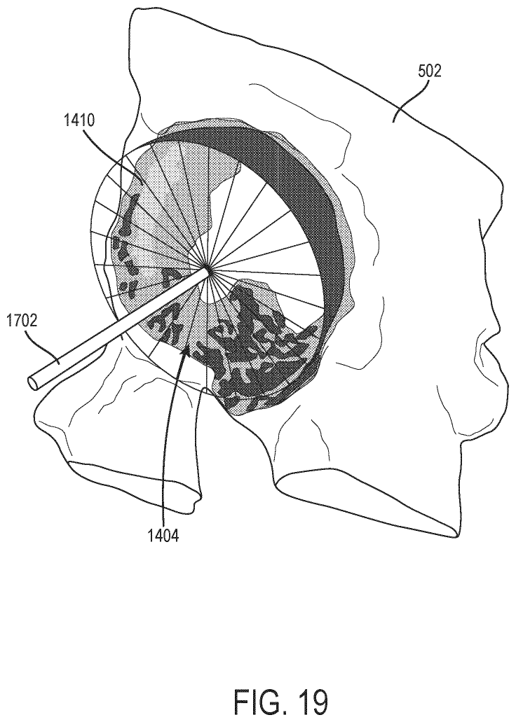

The reamer inserted at step 1802 may have a head with a known geometry (e.g., hemispherical) such that the surgical system 200 has a knowledge of the geometry (including dimensions) of the reamer being used during process 1800 (e.g., based on a user selection of which of a set of reamer heads is being used). The reamer used during process 1800 may also be the reamer used during process 1600, and may be depicted as virtual reamer 1702, described in greater detail below. For example, FIGS. 19-20 and 22-23 each depict the virtual reamer 1702 to represent the reamer used during process 1800. Because the surgical system 200 has the knowledge of the geometry of the reamer being used, the robotic device 220, in combination with the tracking system 222, provides tracking data to indicate where the tool center point of the reamer is relative to the patient's pelvis. Therefore, based on the tool center point and the reamer geometry (i.e., a radius of the reamer head), the surgical system 200 can determine where an outer surface of the reamer head is positioned relative to the bone.

As shown in FIG. 18, process 1800 continues by positioning the reamer within the second cavity such that the head reaches a second bone surface of the second cavity at step 1804. The second bone surface refers to a bone surface that is exposed upon the removal of the first physical implant. Therefore, the head reaches the second bone surface once the head reaches a depth of the second cavity. The second bone surface may be a bone surface of the defect cavity, as described herein. Once the reamer is positioned within the second cavity at step 1804, the position of the reamer is captured at step 1806. In contrast to the single-capture mode used in process 1600, the multi-capture mode used in process 1800 captures the position of the reamer as a user navigates the head of the reamer along the second bone surface of the second cavity. That is, the multi-capture mode captures a plurality of positions of the reamer relative to the second bone surface, rather than capturing a single position of the reamer relative to the second bone surface.

A user may cause the computer system 224 to capture the position of the reamer by clicking, pushing, or otherwise engaging with a foot pedal configured to capture a position of an optically-tracked object (i.e., the reamer) at the moment of engagement. Alternatively or additionally, a user may cause the computing system 224 to capture the position of the reamer by clicking on a graphical user interface when the head of the reamer reaches the second bone surface of the second cavity. Using the multi-capture mode described herein, a user may perform consecutive clicks to cause the computing system 224 to capture the plurality of positions of the reamer.

As shown in FIG. 18, process 1800 continues by identifying a discrepancy between the second bone surface and a first bone surface at step 1808. The first bone surface refers to the bone surface to which the primary cup is coupled, prior to removal during the revision hip arthroplasty procedure. The discrepancy occurs at a point captured by the position of the reamer during step 1806. In some embodiments, the identified discrepancy may correspond to a point along the first bone surface where excess bone was inadvertently removed with the removal of the primary cup during the revision hip arthroplasty procedure. Therefore, a depth of the second cavity (i.e., the defect cavity) may exceed a depth of the first cavity (i.e., the cavity occupied by the primary cup) where excess bone has been inadvertently removed.

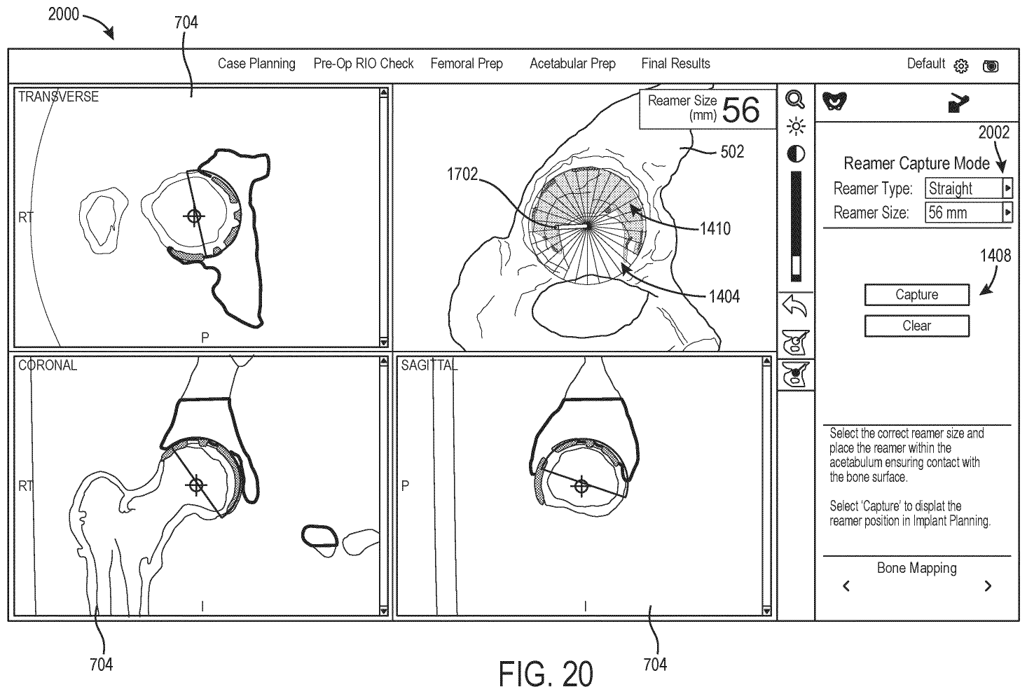

After the discrepancy is identified at step 1808, process 1800 continues with displaying, via a graphical user interface, a virtual model of the second cavity and the second bone surface at step 1810. In some embodiments, the virtual model may be displayed via graphical user interface 2000, as described below with reference to FIGS. 20-24. The virtual model displayed during step 1810 includes a virtual bone surface depicting the discrepancy. For example, FIG. 19 illustrates a virtual model including the virtual bone model 502, the virtual cavity 1404, and the virtual reamer 1702. As shown in FIG. 19, the virtual model also includes the virtual bone surface 1410 depicting the discrepancy between the second bone surface and the first bone surface. The discrepancy is depicted on the virtual bone surface 1410 using a coloration technique to distinguish points/regions where the second bone surface reaches a greater depth (i.e., depicted in a darker shade) than the depth reached at the corresponding points/regions of the first bone surface. In this way, the points/regions where excess bone is inadvertently removed with the primary cup during the revision hip arthroplasty procedure are depicted in a darker shade compared to a remainder of the virtual bone surface 1404. In some embodiments, the virtual bone surface 1410 depicted in FIG. 19 may adopt any other visualization technique to identify the points/regions along the virtual bone surface 1410 where the excess bone has been inadvertently removed with the removal of the primary cup.

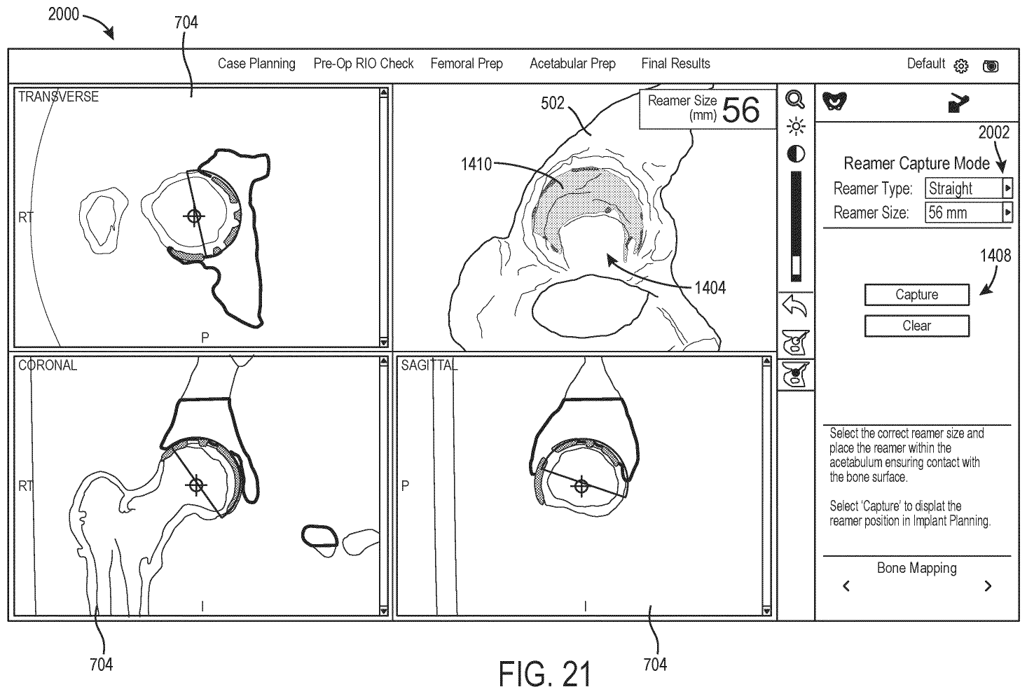

As shown in FIGS. 20-24, the virtual model displayed during step 1810 of process 1800 may be displayed via a graphical user interface 2000. In some embodiments, as shown in FIG. 20, the virtual model includes the virtual bone model 502, the virtual cavity 1404, the virtual bone surface 1410, and the virtual reamer 1702. The virtual model may be illustrated as a 3D model, as shown in FIGS. 20-21, and/or the virtual model may be illustrated using CT images. In some embodiments, the virtual model may be depicted on the graphical user interface 2000 beside one or more CT views 704. FIG. 21 illustrates the virtual model (i.e., the virtual bone model 502, the virtual cavity 1404, and the virtual bone surface 1410) without the virtual reamer 1702.

In some embodiments, as shown in FIGS. 20-21, the graphical user interface 2000 may include the selectable elements 1408. For example, the selectable elements 1408 may include a “Capture” button and a “Clear” button. The “Capture” button may cause the computing system 224 to capture a current position of the reamer relative to the virtual bone model 502 at a time when the “Capture” button is clicked. Similarly, the “Clear” button may cause the computing system 224 to clear the captured position of the reamer relative to the virtual bone model 502. FIGS. 20-21 also illustrate reamer controls 2002 that are used to designate a type (i.e., straight) and a size of the reamer (i.e., 56 mm). The type and the size of the reamer refer to characteristics of the head of the reamer being inserted into the second cavity at steps 1602 and 1802 of processes 1600 and 1800, respectively, such that the geometry of the head being inserted into the second cavity is considered when identifying the discrepancies at steps 1610 and 1808 of processes 1600 and 1800, respectively. The reamer controls 2002 may include a selectable element (i.e., an interactive text box, an arrow, a drop-down menu, etc.) that enable a user accessing the graphical user interface 2000 (i.e., a surgeon performing the revision hip arthroplasty procedure) to inform the surgical system 200 of a change in the type and/or the size of the reamer being used.

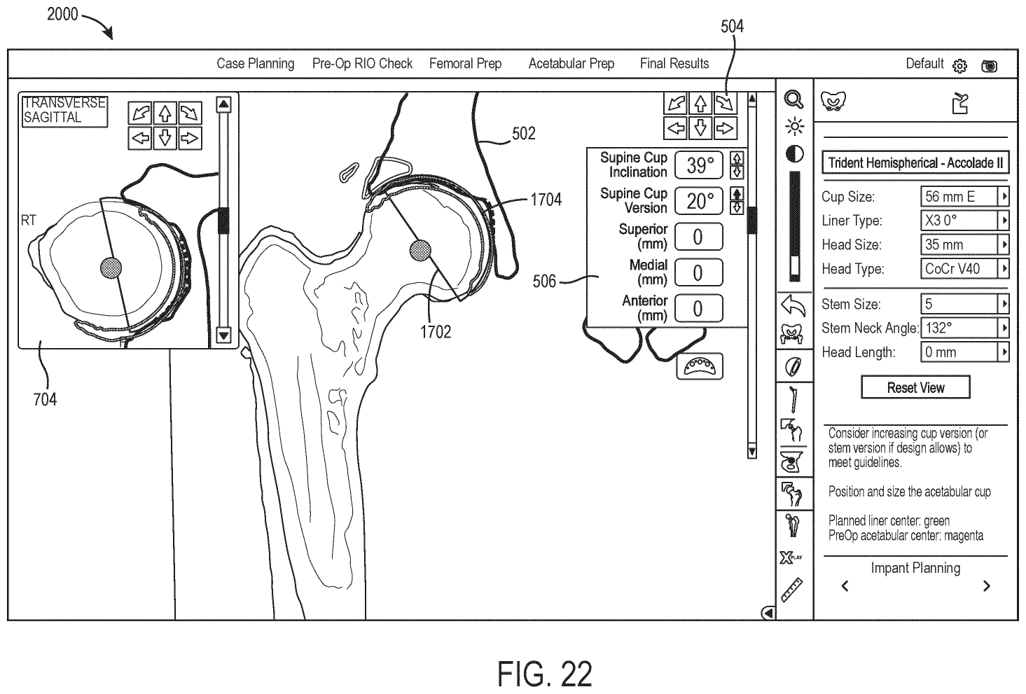

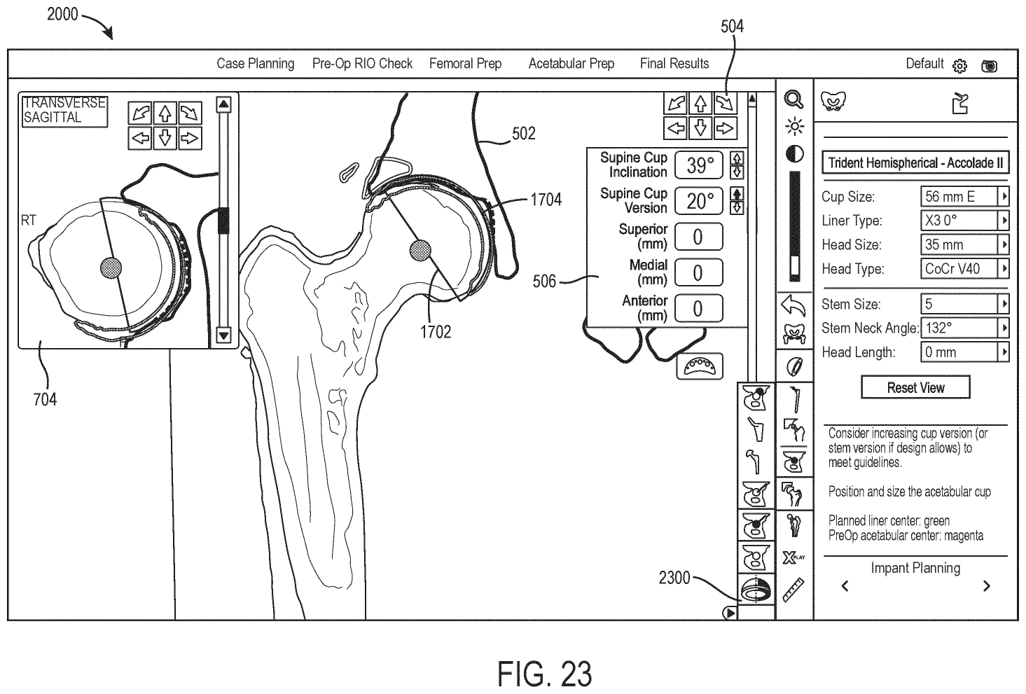

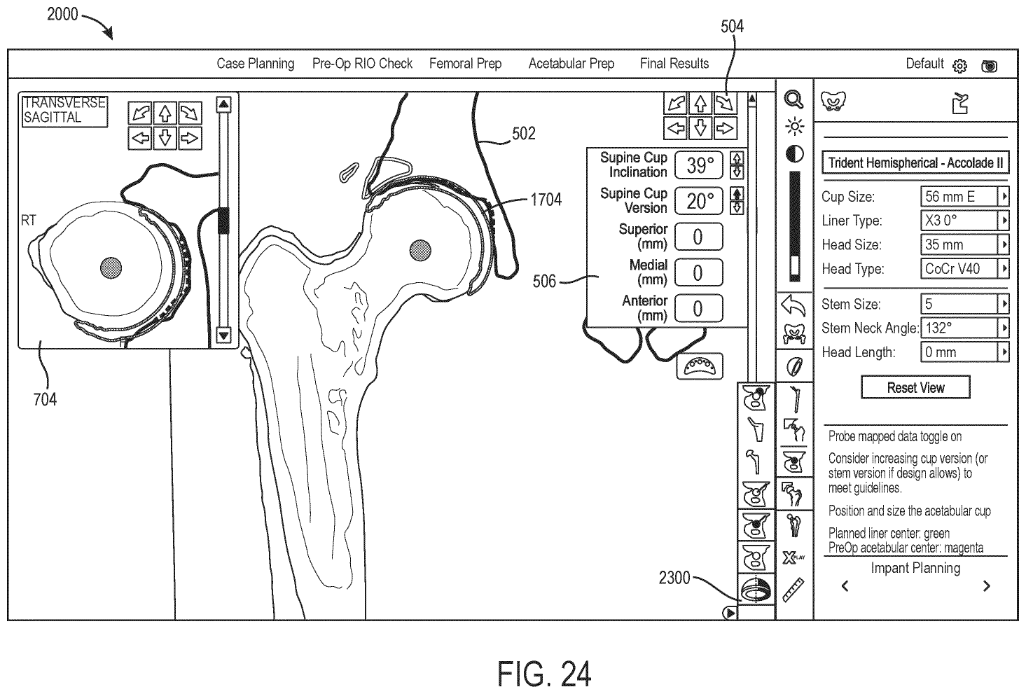

As shown in FIGS. 22-24, the virtual model displayed on graphical user interface 2000 may be depicted as a CT image. FIGS. 22-23 depict the virtual model including the virtual bone model 502, the virtual augment 1702, and the virtual implant 1704, while FIG. 24 depicts the virtual bone model without the virtual augment 1702 (i.e., with the virtual implant 1704 relative to the virtual bone model 502. The graphical user interface 2000 also includes the control arrows 504, the data fields 506, and additional CT views 704. In some embodiments, as shown in FIGS. 23-24, the graphical user interface 2000 may include a retractable tool bar 2300 that enables a user (i.e., a surgeon performing and/or otherwise assisting during a revision hip arthroplasty procedure) to cause the computing system 224 to activate certain functionality of the surgical system 200. FIGS. 23-24 depict the retractable tool bar 2300 while it is expanded, while the retractable tool bar 2300 is collapsed (i.e., not currently depicted) in FIG. 22. In some embodiments, the graphical user interface 2000 may include a selectable element (i.e., an arrow button) configured to cause the computing system 224 to expand and retract the retractable tool bar 2300 on the graphical user interface 2000.

Similar updates such as those made in response to excess bone removal may also be made in response to identification of other features that may be located and registered intra-operatively, for example poor bone stock, cysts, etc. In some embodiments, custom virtual control boundaries are automatically generated intra-operatively based on the tracked positions of a probe moved by a user to positions indicating the location of a feature desired to be resected (e.g., a cyst). The robotic device 220 can then be controlled based on the custom virtual control boundary to resect the identified feature.

Additionally, in some embodiments, the virtual bone model may be updated following an initial resection (e.g., osteophyte resection). For example, a cutting accessory (e.g., attached to the robotic device 220) may be tracked relative to the bone as the cutting accessory is used to remove an osteophyte or other feature. Based on the tracked movement of the cutting accessory, the virtual bone model can be automatically updated to include the modifications made by the cutting accessory by removing the portions of the virtual bone model corresponding to the resected features. The virtual bone model can thereby be updated to accurately represent the post-resection bone surface without reimaging. The surgical plan for remaining steps of the procedure can be updated based on the updated virtual bone model, or other interventions can be planed (e.g., bone graft to fill a void, etc.).

At step 308, the robotic device 220 is controlled to ream the acetabulum to prepare a surface of the pelvis to receive the cup in the planned pose. For example, a virtual control object may be generated based on the planned pose of the cup (referred to herein as the “cup virtual control object”). For example, the cup virtual control object may include a surface corresponding to an exterior surface of the cup and arranged in the planned pose of the cup. Such a surface of the cup virtual control object defines a planned bone modification, e.g., a resulting configuration of the bone after a machining (e.g., reaming) process such that the bone is prepared to receive the cup implant in the planned pose.

The robotic device 220 may be controlled at step 308 using the cup virtual control object. In some embodiments, the robotic device 220 executes autonomous movements as guided by the cup virtual control object to move and operate the surgical tool 234 to ream the pelvis to prepare the pelvis to receive the cup in the planned position. In other embodiments, the robotic device 220 provides haptic feedback to a user to constrain the surgical tool 234 within the cup virtual control object as a user manipulates the surgical tool 234 to ream the pelvis to prepare the pelvis to receive the cup in the planned position. These and other possible control modalities are described in detail above with reference to FIG. 2.

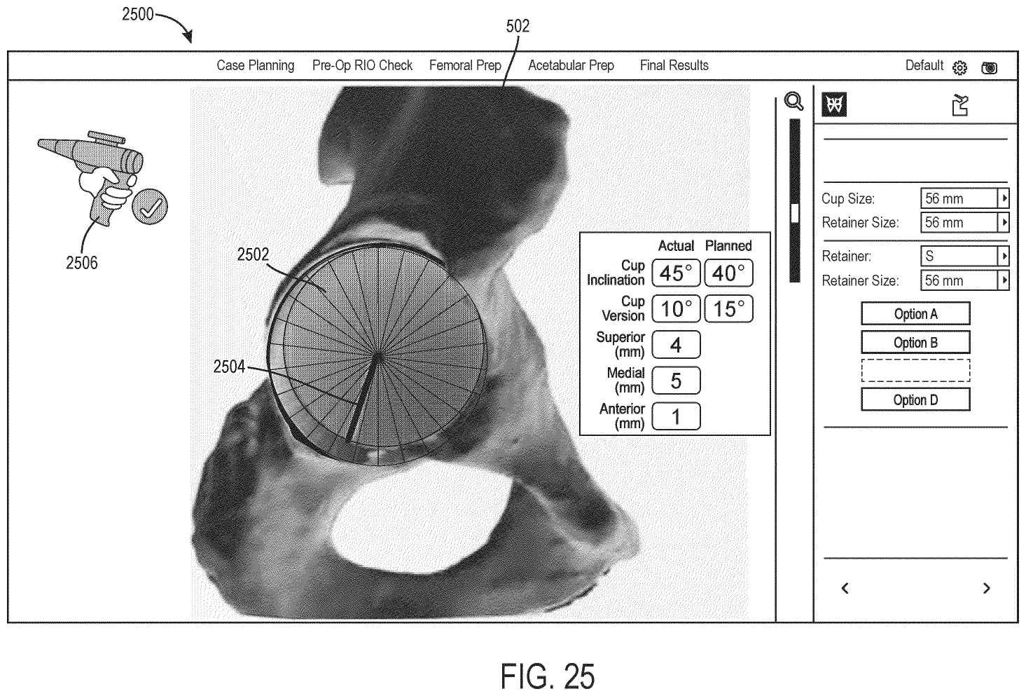

FIG. 25 shows an example of a graphical user interface 2500 that may be generated by the processing circuit 260 and displayed on the display 264 to facilitate execution of step 308, for example an in embodiment where the robotic device 220 is a haptic device. The graphical user interface 2500 shows the virtual bone model 502 with a color-coded (e.g., green) or shaded region 2502 indicating areas of the bone that are to be removed in accordance with the surgical plan. An arrow 2504 indicates a current orientation and center point of the surgical tool 234. A tool indicator 2506 indicates that the surgical tool 234 is currently operating (e.g., that the reamer is rotating).

The processing circuit 260 is configured to update the graphical user interface 2500 in real time using the tracked poses of the pelvis and the surgical tool 234 from the tracking system 222. For example, the color-coded or shaded region 2502 may be reduced in size as the tracking data indicates that the cutting accessory of the surgical tool 234 (e.g., the head of a reamer tool) passes through the corresponding area of the bone. Completion of the planned bone modification corresponds to full consumption (reduction to nothing, erasure, etc.) of the color-coded or shaded region 2502.

The virtual control object may also be indicated on the graphical user interface 2500. In some cases, the processing circuit 260 may provide a different color-coding (e.g., red) to indicate areas where data from the tracking system 222 indicates that surgical tool 234 violated the constraints of the virtual control object and modified the bone beyond the surgical plan.

At step 310, the robotic device 220 is controlled to ream the acetabulum to prepare a surface of the pelvis to receive the implant augment in the planned pose of the implant augment.

For example, a virtual control object may be generated based on the planned pose of the augment (referred to herein as the “augment virtual control object”). For example, the augment virtual control object may include a surface corresponding to an exterior surface of the augment and arranged in the planned pose of the augment. Such a surface of the augment virtual control object defines a planned bone modification, e.g., a resulting configuration of the bone after a machining process such that the bone is prepared to receive the augment implant in the planned pose.

In some embodiments, the cup virtual control object and the augment virtual control object are separate virtual control objects and are applied sequentially to execute the surgical plan by first preparing the bone to receive the cup and then preparing the bone to receive the augment. In some cases, the sequence may be reversed, such that the robotic device 220 is controlled to first prepare the bone to receive the augment using the augment virtual control object and then the cup virtual control object is applied to control the robotic device 220 to prepare the bone to receive the cup.

In some such embodiments, a different approach orientation for the surgical tool may be required by the cup virtual control object and the augment virtual control object. The processing circuit 260 may determine completion of the first bone modification (i.e., an end of step 308) and guide the surgical tool from the orientation required by the cup virtual control object into the orientation required by the augment virtual control object, for example using a collapsing haptic boundary, before initiating the second bone modification (i.e., execution of step 310). Additionally, in some embodiments, a change to the surgical tool 234 may be made between steps 308 and 310, for example such that a first reamer head with a first size is used to prepare the cup region and a second reamer head with a second (e.g., smaller) size is used to prepare the bone to receive the augment. The graphical user interface 2500 may display a prompt to make such a change to the surgical tool 234.

In other embodiments, the cup virtual control object and the augment virtual control object are combined as a single virtual control object that includes surfaces corresponding to both the cup and the augment. In such embodiments, steps 308 and 310 can be executed in a unified (simultaneous) manner.

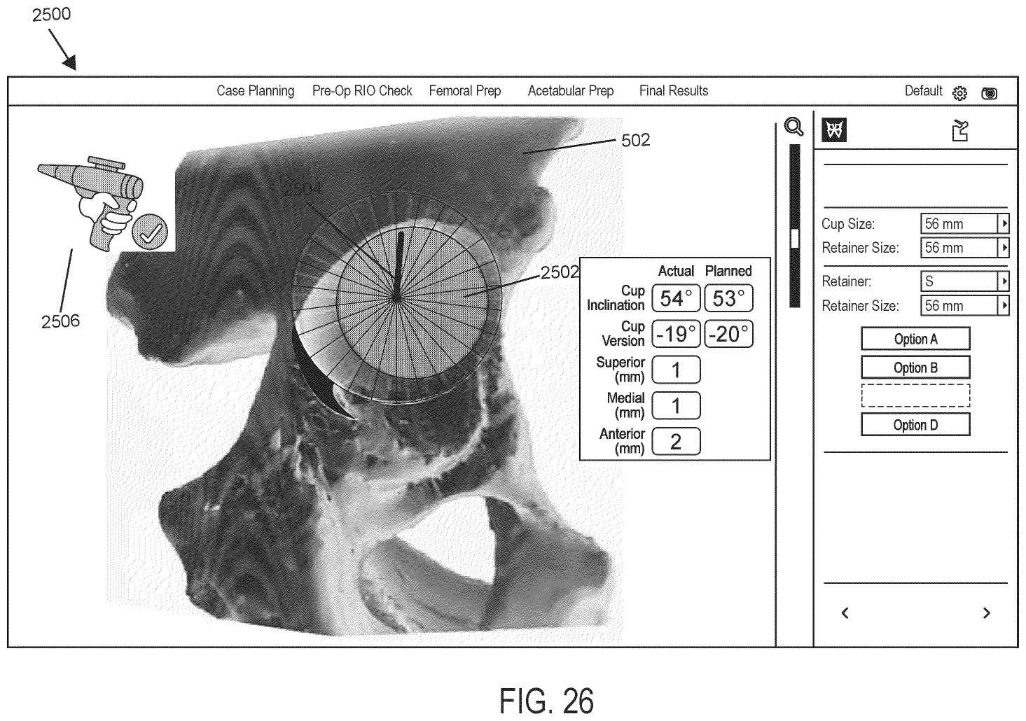

FIG. 26 shows the graphical user interface 2500 as displayed during step 310 in an exemplary embodiment. The graphical user interface 2500 shows the virtual bone model 502 with a color-coded (e.g., green) or shaded region 2502 indicating areas of the bone that are to be removed in accordance with the surgical plan during step 310. The virtual bone model 502 has been modified by the processing circuit 260 to visualize the modifications to the actual bone made during step 308. An arrow 2504 indicates a current orientation and center point of the surgical tool 234. In the example shown, the arrow 2504 has changed orientation relative to the orientation of the arrow 2504 as shown in FIG. 25. A tool indicator 2506 indicates that the surgical tool 234 is currently operating (e.g., that the reamer is rotating).

To facilitate step 308, the processing circuit 260 is configured to update the graphical user interface 2500 in real time using the tracked poses of the pelvis and the surgical tool 234 from the tracking system 222. For example, the color-coded or shaded region 2502 may be reduced in size as the tracking data indicates that the cutting accessory of the surgical tool 234 (e.g., the head of a reamer tool) passes through the corresponding area of the bone. Completion of the planned bone modification corresponds to full consumption (reduction to nothing, erasure, etc.) of the color-coded or shaded region 2502.

Steps 308 and 310 thereby result in a bone (e.g., pelvis) prepared to receive the cup in the pose planned at step 302 and to receive the implant in the pose planned at step 304.

At step 312, the augment is placed in the planned pose and a match between the actual pose of the augment and the planned pose is verified, for example as illustrated in the example embodiment of FIG. 27. As shown in FIG. 27, a surgeon has manually placed the augment 2700 in the surgical site and adjacent the bone in approximately the planned pose. A navigation probe 2702 is shown as touching a point on the augment 2700. The navigation probe 2702 can be tracked by the tracking system 222, such that the tracking system 222 can ascertain a location of the tip 2704 of the probe 2702 relative to other tracked objects, for example the bone modified at steps 308-310. By tracking the navigation probe 2702 as the navigation probe 2702 is touched to multiple points on the augment 2700, a pose of the augment 2700 can be determined by the tracking system 222 and the processing circuit 260. In such embodiments, the processing circuit 260 is configured to compare the tracked pose of the augment 2700 to the planned pose of the augment from step 304. The processing circuit 260 may cause the display 264 to display an indication that the tracked pose of the augment 2700 matches the planned pose of the augment and/or provide guidance for modifying the actual pose of the augment 2700 to bring the tracked pose of the augment 2700 into agreement with the planned pose of the augment 2700. In other embodiments, the augment 2700 may be coupled to a tracked inserter tool, such that the processing circuit can use the tracked pose of the inserter tool to facilitate navigation of the augment to the planned pose. In some embodiments, the inserter tool is supported by the robotic device 220 or another robotic arm such that the inserter tool can hold the augment 2700 in a selected position.

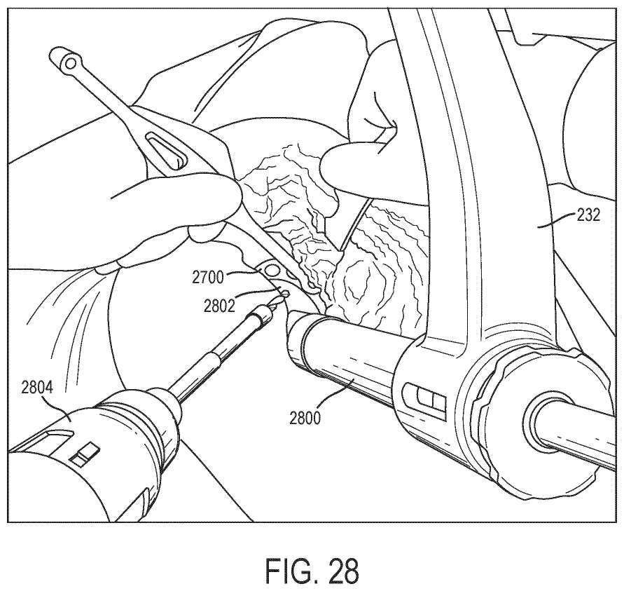

At step 314, the robotic device 220 is controlled to hold the augment in the planned placement while the augment is coupled to the pelvis, for example as illustrated in the example embodiment of FIG. 28. As shown in FIG. 28, the augment 2700 is positioned as described with reference to FIG. 27 and step 312. A holder arm 2800 is coupled to the robotic arm 232 and is shown as holding a trial cup implant 2802. The robotic arm 232 is controlled to force the trial cup implant 2802 against the augment 2700 to push the augment 2700 against the bone, thereby holding the augment 2700 in the planned pose relative to the bone. The augment 2700 can then be coupled to the bone. In the example of FIG. 28, a surgical drill 2804 (e.g., a flexible drill) is used to insert one or more screws through the augment 2700 and into the bone to secure the augment 2700 to the bone in the planned position. The trial cup implant 2802, as held in position by the robotic device 220, can substantially prevent movement of the augment 2700 while the screws are inserted, thereby reducing the number of surgeons or surgical assistants needed to conduct the surgery, improving visibility of the surgical field, and improving accuracy of placement of the augment 2700 relative to the surgical plan. Although a trial cup implant is used in this embodiment, a final cup implant may also be used in step 314.

In other embodiments, at step 314, the augment 2700 is coupled to the holder arm such that the holder arm can be moved by the robotic device 220 to adjust the position of the augment 2700. In such an embodiment, the robotic device 220 is controlled to move the augment 2700 to the planned pose, for example autonomously or by providing haptic feedback to a surgeon. In some embodiments, the surgical drill 2804 is robotically-controlled (e.g., coupled to a second robotic arm) and configured to autonomously insert screws through the augment into the bone in accordance with a surgical plan. In some embodiments, a cutting accessory of surgical tool 234 can be used (autonomously or under haptic guidance) to prepare pilot holes for screw insertion. In some such embodiments, a screw insertion accessory can then be mounted to surgical tool 234 to insert (autonomously or under haptic guidance) bone screws into the pilot holes and through the augment.

At step 316, the implant cup is placed in substantially the planned pose for the implant cup (e.g., slightly spaced from the planned pose in anticipation of step 320 described below). In some embodiments, the cup is manually positioned by a surgeon and that position is checked using a navigation probe as described above for the augment with reference to step 312. In other embodiments, the implant cup is mounted on an impaction arm coupled to the robotic device 220. The robotic device 220 is controlled to move the implant cup to substantially the planned pose, for example autonomously or by providing haptic feedback to a user. For example, haptic feedback may be provided by constraining the position of the implant cup within a virtual control object that collapses (gets smaller, converges) as the implant cup is brought closer to the planned pose, i.e., such that the implant cup can be moved closer to the planned pose but not substantially further away from the planned position relative to a current position. The implant cup is thereby positioned and oriented in substantially the planned pose.

At step 318, cement is provided between the cup and the augment. As mentioned above with reference to step 304, the planned pose of the augment is spaced apart from the planned pose of the cup to allow for cement to be included between the cup and the augment to couple the cup to the augment. By following steps 312-316, the actual positions of the cup and the augment also provide space for cement between the cup and the augment. Accordingly, process 300 facilitates use of a predictable, consistent, and preferred (planned, clinically-validated, etc.) amount of cement between the cup and the augment.

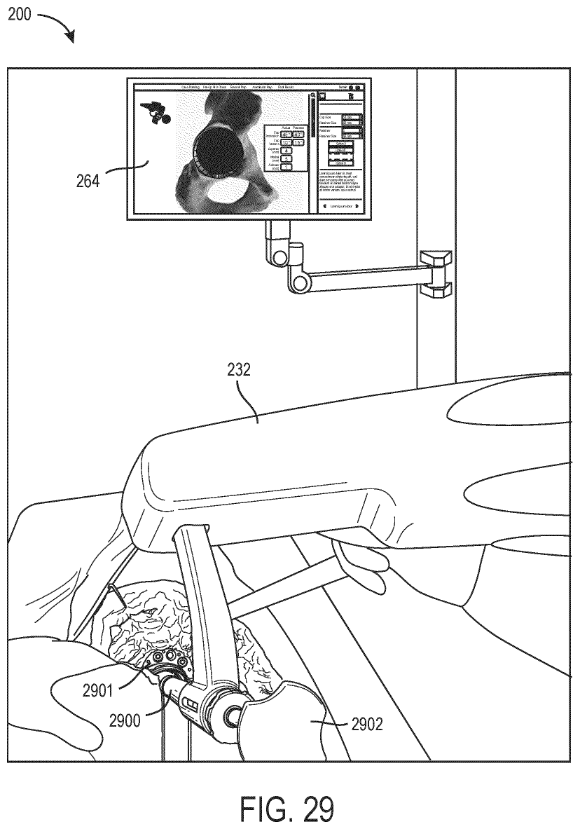

At step 320, the robotic device is controlled to facilitate cup impaction to fix the cup in the planned placement. FIG. 29 shows an example embodiment of the surgical system 200 arranged to execute step 320. As shown in FIG. 29, an impaction device 2900 is mounted on the robotic arm 232. The robotic arm 232 is controlled to align the impaction device 2900 with the planned orientation of the cup and such that a distal end 2901 of the impaction device 2900 is in contact with the cup at substantially the planned position for the cup. FIG. 29 shows the display device 264 as providing an indication that the impaction device 2900 is properly positioned for cup impaction. When the surgical system 200 is in the state shown in FIG. 29, the surgeon may provide a blunt force to a proximal end 2902 of the impaction device 2900. The force is transmitted along the impaction device 2900 to impact the cup into the pelvis. This force causes the cup to be driven into the pelvis to substantially fix the cup relative to the pelvis. The robotic arm 232 and information displayed on the display device 264 facilitates a surgeon in accomplishing impaction such that the cup is fixed to the pelvis in the planned pose (i.e., as planned at step 302).

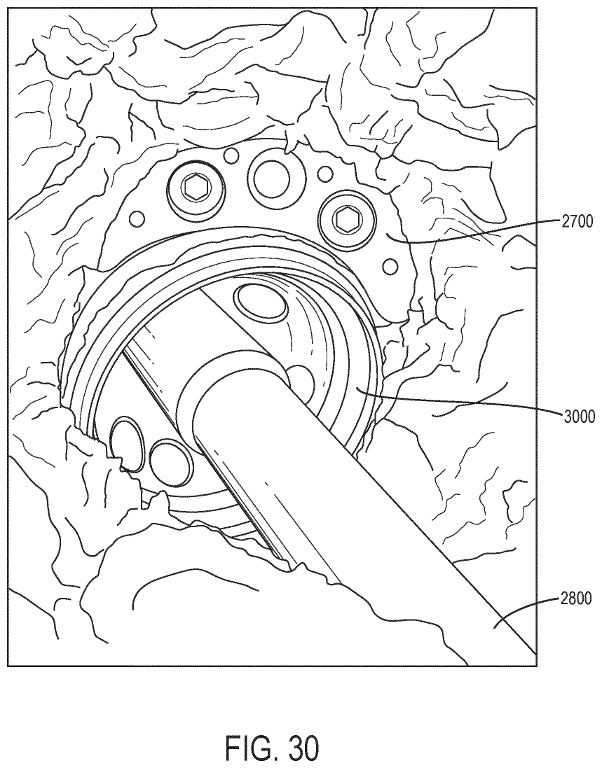

At step 322, the robotic device is controlled to continue to hold the cup in the planned pose for the duration of cement curing (e.g., ten minutes). FIG. 30 illustrates step 322 in an example embodiment, and shows the implant cup 3000 held in position relative to the implant augment 2700 by a holder arm 2800. The holder arm 2800 may be the same device as the impaction device 2900 or a different device. By automating this holding task, a surgeon or surgical assistant may advantageously become free to accomplish other tasks relating to the surgical procedure. Additionally, robotically-assisted and tracked positioning during cement curing may ensure that the planned geometric relationship between the cup and the augment is achieved. Furthermore, integrity of the cement mantle and unitization of the cup and augment may be optimized because relative movement is minimized as the cement hardens.

Following step 322, the surgical procedure may proceed following established workflows, for example to position a liner in the cup, to position a femoral implant in the cup, to repair soft tissue proximate the hip joint, and to close the surgical incision. The surgical system 200 may be configured to assist with some or all of these additional steps in various embodiments. Process 300 may thereby improve surgical efficiency and experience for surgeons, reduce the duration of a surgical procedure, and improve patient outcomes by providing accurate placement of augments and cups in accordance with personalized surgical plans.

In some embodiments, data is collected relating to the planning and procedures conducted using the systems and methods described herein. For example, details such as the types of implants used, bone density, ligament balancing measurements, final implant placement (angle, anterior/posterior placement, medial/lateral placement, placement with respect to a joint line, mechanical and anatomic axis positions, etc.), among other possibilities, can be collected during planning of the procedures. Post-operative outcomes may also be collected. The post-operative outcomes may then be compared to the other data to provide insights into improved execution and implementation of the systems and methods described herein.

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, use of materials, colors, orientations, etc.). For example, the position of elements may be reversed or otherwise varied and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions and arrangement of the exemplary embodiments without departing from the scope of the present disclosure.

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and are considered to be within the scope of the disclosure.

What is claimed is:

1. A method, comprising:
obtaining a surgical plan comprising a removal of a first physical implant from a bone and a replacement of the first physical implant with a second physical implant;
receiving a first virtual model comprising a first virtual implant model relative to a first virtual bone surface of the bone, wherein the first virtual bone surface represents a first bone surface to which the first physical implant is coupled;
identifying a second bone surface resulting from the removal of the first physical implant from the bone by tracking a position of a surgical tool relative to the bone;
detecting a discrepancy between the first bone surface and the second bone surface; and
displaying, on a graphical user interface, a visualization of the discrepancy.

2. The method of claim 1, further comprising:
identifying a first cavity occupied by the first physical implant, wherein the first bone surface reaches a first depth of the first cavity; and
identifying a second cavity exposed by the removal of the first physical implant, wherein the second bone surface reaches a second depth of the second cavity.

3. The method of claim 2, further comprising updating the surgical plan such that the second physical implant occupies the second cavity.

4. The method of claim 3, wherein updating the surgical plan further comprises at least one of updating the second physical implant or inserting an augment at a position of the discrepancy.

5. The method of claim 2, wherein detecting the discrepancy comprises determining that the second depth exceeds the first depth at one or more positions along the second bone surface.

6. The method of claim 2, wherein the surgical tool comprises a reamer held by a robotic arm, the method comprising controlling the robotic arm to enable insertion of the reamer into the second cavity.

7. The method of claim 6, wherein the reamer detects the discrepancy using a single capture mode, the method comprising:
capturing a position of the reamer relative to the second bone surface; and
displaying a visualization of the reamer over the first virtual model.

8. The method of claim 6, wherein the reamer detects the discrepancy using a multi-capture mode, the method comprising:
controlling the robotic arm to move the reamer across a plurality of positions relative to the second bone surface;
capturing the plurality of positions of the reamer relative to the second bone surface while the reamer moves across the plurality of positions; and
displaying a visualization of the plurality of positions of the reamer over the first virtual model.

9. The method of claim 1, wherein the surgical tool comprises a probe, wherein the method comprises optically tracking the probe as the probe contacts the second bone surface.

10. A system comprising:
a robotic device; and
circuitry programmed to:
obtain a surgical plan comprising a removal of a first physical implant from a bone and a replacement of the first physical implant with a second physical implant;
receive a first virtual model comprising a first virtual implant model relative to a first virtual bone surface of the bone, wherein the first virtual bone surface represents a first bone surface to which the first physical implant is coupled;
identify a second bone surface resulting from the removal of the first physical implant from the bone by tracking a position of a surgical tool relative to the bone;
detect a discrepancy between the first bone surface and the second bone surface; and
display, on a graphical user interface, a visualization of the discrepancy.

11. The system of claim 10, wherein the circuitry is further programmed to:
identify a first cavity occupied by the first physical implant, wherein the first bone surface reaches a first depth of the first cavity; and
identify a second cavity exposed by the removal of the first physical implant, wherein the second bone surface reaches a second depth of the second cavity.

12. The system of claim 11, wherein the circuitry is further programmed to update the surgical plan such that the second physical implant occupies the second cavity.

13. The system of claim 12, wherein updating the surgical plan further comprises at least one of updating the second physical implant or inserting an augment at a position of the discrepancy.

14. The system of claim 11, wherein detecting the discrepancy comprises determining that the second depth exceeds the first depth at one or more positions along the second bone surface.

15. The system of claim 11, wherein the surgical tool comprises a reamer held by a robotic arm, the circuitry further programmed to control the robotic arm to enable insertion of the reamer into the second cavity.

16. The system of claim 15, wherein the reamer detects the discrepancy using a single-capture mode, the circuitry further programmed to:
capture a position of the reamer relative to the second bone surface; and
display a visualization of the reamer over the first virtual model.

17. The system of claim 15, wherein the reamer detects the discrepancy using a multi-capture mode, the circuitry further programmed to:
control the robotic arm to move the reamer across a plurality of positions relative to the second bone surface;
capture the plurality of positions of the reamer relative to the second bone surface while the reamer moves across the plurality of positions; and
display a visualization of the plurality of positions of the reamer over the first virtual model.

18. The system of claim 10, wherein the surgical tool comprises a probe, wherein the circuitry is further programmed to optically track the probe as the probe contacts the second bone surface.

19. One or more non-transitory computer-readable media storing instructions that, when executed by a processor, cause the processor to perform operations comprising:
obtaining a surgical plan comprising a removal of a first physical implant from a bone and a replacement of the first physical implant with a second physical implant;
receiving a first virtual model comprising a first virtual implant model relative to a first virtual bone surface of the bone, wherein the first virtual bone surface represents a first bone surface to which the first physical implant is coupled;
identifying a second bone surface resulting from the removal of the first physical implant from the bone by tracking a position of a surgical tool relative to the bone;
detecting a discrepancy between the first bone surface and the second bone surface; and
displaying, on a graphical user interface, a visualization of the discrepancy.

20. The one or more non-transitory computer-readable media of claim 19, wherein the operations further comprise:
identifying a first cavity occupied by the first physical implant, wherein the first bone surface reaches a first depth of the first cavity; and
identifying a second cavity exposed by the removal of the first physical implant, wherein the second bone surface reaches a second depth of the second cavity.
